(19) United States
(12) Patent Application Publication
(10) Pub. No.: US 2007/0072179 A1
(43) Pub. Date: Mar. 29, 2007

(54) Solid-phase assisted spectroscopic and spectrometric analysis of complex biopolymer mixtures

(76) Inventors: Frank Essmann, Erkrath (DE); Christian Scheler, Berlin (DE); Sascha Thies, Berlin (DE)

Correspondence Address: NEEDLE & ROSENBERG, P.C., SUITE 1000, 999 PEACHTREE STREET, ATLANTA, GA, 30309-3915, US

(21) Appl. No.: 10/514,052

(22) PCT Filed: May 9, 2003
(86) PCT No.: PCT/EP03/04878

§ 371 Date: Aug. 11, 2005

(30) Foreign Application Priority Data
May 10, 2002 (DE) 102 20 804.2
May 10, 2002 (EP) 02010555.7

(51) Int. Cl. C12Q 1/68; G01N 33/53; C12Q 1/37; C12M 1/34

(52) U.S. Cl. 435/6; 435/7.1; 435/23; 435/287.2

(57) ABSTRACT

The present invention relates to a method and kit for analyzing complex mixtures of biopolymers from one or more samples.

### FIELD OF THE INVENTION

The present invention relates to a method and kit for analyzing complex mixtures of biopolymers from one or more samples.

### BACKGROUND OF THE INVENTION

During the search for molecular causes of various diseases, molecular biology/DNA analysis has reached a point where it is possible to read out the whole genomic information even for complex organisms. Accordingly, the completion of the human genome project has come within our reach, and the sequencing of the genomes of other organisms has already been completed. Further, the mRNA expression for an immense number of genes can be quantified simultaneously. By means of DNA array technology, it is even possible to establish the influence of environmental conditions on the expression level of genes. This means that qualitative and quantitative information about genes and gene expression in organisms and tissues can be obtained at least in theory.

This immense amount of accessible and existing data requires an intelligent and efficient management. For this reason, the introduction of fast and powerful computers and the development of intelligent software has been a precondition for the handling of such enormous amounts of data. In the meantime, the handling of enormous amounts of data has been implemented in routine operation, both in terms of structural elucidation and biochemical interactions, regulation and function of genes/proteins. Unfortunately, it is all the same within the nature of genetic information that it does not allow any conclusions on regulation mechanisms or the expression level of proteins.

In accordance with the generally accepted dogma of biology, proteins are the active components of biological systems while the DNA is merely a storage medium for the information needed for the production of proteins. Certain types of RNA function as a link between the DNA memory of information and the functioning components, i.e., the proteins, either by translating the information contained in the DNA into a convertible form, or as carriers of smallest units of information, coupled to the basic components of proteins (amino acids).

No biological system is known in which this direction of the flow of information is reverted. For this reason, the quantity of analyte available for protein-analytic methods is generally very much limited. Therefore, the currently employed method for the identification and quantification of all proteins represented in a system under particular environmental conditions necessarily must be extremely sensitive. Further, proteins may be subjected to post-translational modification, which can affect, for example, their half life, biological function and/or activity. Such post-translational modifications include, for example, phosphorylation, glycosylation, farnesylation, the binding of nucleotides and metal ions, just to mention a fraction of the possibilities. All these pieces of information are not available from genetic information alone.

The mentioned complete description of the whole protein content of a biological system under defined conditions including the expression level, modifications and identity of the proteins is called a proteome. After the complete sequencing of the human genome, proteome analysis is the forthcoming milestone of life sciences, and for the reasons and due to the limitations mentioned above, it is an incomparably more complex and more comprehensive object. For these reasons, it appears essential to reduce the complexity of the samples to be analyzed to a degree which can be handled with the available means without reducing the contained information.

As described above, there is no method of protein analysis which has a sensitivity comparable to that of DNA/RNBA analytics and/or a comparable through-put, since proteins cannot be amplified in vitro. Currently, two basically different technologies for the analysis of complex biological mixtures are being used. These are two-dimensional gel electrophoresis on the one hand and multidimensional liquid chromatography, which is still in its infancy, on the other hand. A third method, the so-called ICAT method, makes use of specific chemical components which introduce a (non-)radioactive affinity label into biopolymers (WO 00/11208).

Two-dimensional gel electrophoresis (2DE) is the most wide-spread technology for proteome analysis. The complex mixture of proteins is separated in two dimensions in a homogeneous polymer matrix. Thus, the complex mixture of biopolymers (proteins) is first separated on the basis of the isoelectric point of the components. The proteins thus separated in the matrix are subsequently separated by their apparent molecular weight in an analogous matrix. The subsequent staining results in a specific dot pattern in which each dot ideally corresponds to one protein.

For the two-dimensional gel-electrophoretic separation of proteins, two different methods are currently employed. These are NEPHGE (non-equilibrium pH gradient electrophoresis) as published by J. Klose (Humangenetik 26(3), 231-243 (1975)) and O'Farel and IPG technology as developed by A. Görk et al. (Electrophoresis 9(1), 57-59 (1988)). The two methods are distinct mainly in the performance of the first dimension (isoelectric focusing). Thus, a pH gradient is established in which the proteins will migrate due to their charge, after an electric field has been applied, to the point where their charge is zero. At this point, no electric force will act on the proteins, and they remain there in the PAA matrix. The pH gradient is either established by mobile ampholytes (NEPHGE) or introduced into the gel by polymerization when the matrix is prepared (IPG technique). However, this method is not limited to PAA matrixes, but is compatible with other materials (e.g., agarose). Further, there are systems which allow isoelectric focusing to be performed in solution.

The separation matrix, which contains the one-dimensionally separated proteins, is now transferred onto another matrix in which the further separation (2nd dimension) is effected. The latter usually consists of an SDS-PAGE (sodium dodecylsulfate polyacrylamide gel electrophoresis). The proteins are thereby separated in the matrix by their apparent molecular weight.

The whole procedure is followed by staining which renders the proteins/polymers visible. This results in a sample-specific dot pattern which is subjected to a detailed comparative analysis.

Multidimensional liquid-phase chromatography (MDLC) is a method not employed in analytics as a matter of routine, and its spreading is far behind that of 2DE. However, it has various advantages over 2DE. The complex protein/biopolymer mixtures are separated on the basis of specific interactions with the surface of the separation material. Depending on their individual compositions, the biopolymers exhibit a specific retention time.

After the separation has been effected, the separated biopolymers must be identified (protein identification). In the case of 2DE, the procedure is as follows. After staining, the interesting spots are cut out of the matrix and decomposed to smaller fragments by means of enzymatic or chemical reaction. The fragments can diffuse from the matrix and are subsequently subjected to mass-spectroscopic analysis. Thus, the masses of the produced fragments are established, wherefrom an identification (unequivocal assignment to a data set of a protein data base) can be achieved in connection with other known data on the respective analyte. If the analysis is effected in a device with MS/MS capability, a fragmentation of a specific fragment can be performed and its composition thus established more precisely. From knowledge on the composition of one or more existing fragments, it is possible to achieve the identification of the starting analyte with higher probability. The sequential approach in this analysis is the limiting step in the identification of the proteins. In some cases, mass spectrometers require only a few fmol of an analyte for analysis. However, since the greatest losses occur during the preparation of the biopolymer for MS analysis, a far greater quantity of starting material must be available.

Most recently, the development of methods and instruments for the automated and data-dependent mass-spectrometric analysis in connection with microcapillary electrophoresis has substantially improved the sensitivity and speed of protein gel separation as well as the analysis of previously fragmented complex mixtures of biopolymers. In this respect, the identification of proteins has made significant progress, whereas the (relative) quantification thereof is still extremely problematic.

The dynamic range of a wide variety of staining and detection methods available for the quantification of the proteins does not cover the required orders of magnitude. In a single cell, proteins can be present in numbers of copies ranging from 1 to several million, which illustrates the problems. In 2DE, the following staining methods are generally employed:

| Method | Detection limit/ng |
|---|---|
| Silver with glutardialdehyde¹ | 10 |
| Silver² | 1 |
| Zinc/imidazole | 100 |
| KCl | 100 |
| Coomassie R250 | 100 |
| Colloidal coomassie G250 | 30 |
| Fluorescence | 10 |

¹no further processing (identification) possible
²suitable for subsequent mass-spectrometric analysis

In the relevant field of technology, very few patents/patent applications exist:

WO 00/11208 describes the ICAT method by which one or more proteins or protein functions in one or more samples can be identified, so that a qualitative and quantitative analysis of expression profiles becomes possible. Thus, a labeling reagent is employed which has a different isotope labeling for each sample, which enables the quantitative determination of relative amounts of proteins in different samples. Further, the labeling reagent includes an affinity label, a linker and a protein-reactive group which will react either with a functional group of a protein or as a substrate for an enzyme. Each sample is admixed with a labeling reagent, and affinity-tagged proteins or enzyme products are prepared which are then captured with capture reagents which selectively bind the affinity label. After the affinity-tagged components have been released, the detection and identification of the released affinity-tagged components is effected by mass spectrometry. However, one drawback of this method is its limitation to proteins and to isotope labeling as the only labeling which ensures assignment of the respective protein to the starting sample and enables the quantitative determination of relative amounts of proteins from various samples. Further, the method disclosed in WO 00/11208 does not enable sequencing, i.e., the determination of the primary structure of proteins, or a reduction of the number of samples by combining similar molecules prior to the mass-spectrometric examination.

EP-A-1 106 702 relates to a high-throughput screening method for detecting non-covalent interactions between one or more test compounds and polynucleotides, the polynucleotides being in an equilibrium between single-stranded and double-stranded forms. The complexes of test compound and polynucleotide formed in solution due to non-covalent interactions are then examined by means of ESI-MS (electrospray ionization mass spectrometry). Thus, the method of EP-A-1 106 702 is limited to the detection of compounds which interact with polynucleotides, but is not concerned with the structural elucidation of the polynucleotides themselves or other biomolecules. Finally, a reduction of the number of samples before the ESI-MS examination is not provided. A method which combines liquid chromatography with ESI-MS (LC-ESI-MS analysis) has been disclosed in U.S. Pat. No. 6,139,734, in which the separation of the compounds to be tested, especially biologically relevant compounds, by means of high-performance liquid chromatography (HPLC) is effected with controlling the flow rate of the mobile phase in the HPLC column. Thus, the method of U.S. Pat. No. 6,139,734 also does not enable the identification of biomolecules or reduction of the number of samples prior to the ESI-MS examination.

WO 02/29414 describes a method in which one or more biomolecules (e.g., proteins, peptides) in one or more samples are subjected to unique mass tagging.

After said unique tagging, the different samples are combined, their components (biomolecules) are separated, for example, by chromatography, and the individual fractions are measured in a mass spectrometer. Due to the mass tagging, the biomolecules can be quantified and assigned to the individual samples.

WO 00/67017 describes a method for the in vivo isotope labeling of proteins in biological material. A sample culture is incubated with a (nutrient) medium which contains a particular isotope. Another culture is incubated with a different isotope. Both samples, which thus have different isotope labels, are combined, the proteins are extracted and fractioned by chromatographic or other methods. The individual fractions may then be analyzed and relatively quantified by mass spectrometry. Due to the isotope labeling, the biomolecules can be assigned to their sample of origin.

In addition, the following relevant publications exist:

M. B. Smolka et al., Anal. Biochem., 297(1): 25-31 (2001), describes the systematic optimization of the ICAT method with variation of the concentrations of various chemicals (e.g., SDS, urea) and reaction conditions (duration). A specific and quantitative labeling of the analytes is demonstrated.

T. J. Griffin, J. Am. Soc. Mass Spectrom., 12(12): 1238-46 (2001), describes an improvement of the ICAT method. For increasing the effectiveness, the peptides which are present in different quantities in the samples to be compared are identified first. Only the differently represented peptides are subjected to a detailed mass-spectrometric analysis (fragmentation and MS/MS). In this connection, a corresponding software which automates the analysis described is presented.

D. K. Han et al., Nat. Biotechnol. October, 19(10): 946-51 (2001), describes a special application of ICAT technology. The first step is the preparation of the microsomal fractions to be compared. These are subsequently provided with the isotope-labeled affinity tag, mixed and digested enzymatically. This is followed by a multidimensional chromatographic separation of the resulting peptide mixtures. The chromatographic separation directly leads into a tandem mass spectrometer which enables both the analysis of the relative intensities of two peptide peaks and the establishing of sequence information about them. The authors describe the application of the method for the identification and relative quantification of 491 proteins in native and in vitro differentiated HL-60 cells.

F. Turecek, J. Mass Spectrom., 37: 1-14 (2002), gives a survey of applications of ICAT technology as well as the related ACESIMS technology. Both methods are described in detail, and examples of their application for the diagnosis of, for example, GM1 or mucopolysaccharidosis type III (Sanfilippo syndrome type A-d) are provided.

In M. B. Goshe et al., Anal. Chem., 73(11): 2578-86 (2001), the selective purification and enrichment of 0-phosphorylated peptides is described. The method is a slightly modified version of the ICAT method. After β-elimination by hydroxide, a functionalized linker reagent in a deuterated or non-deuterated form was added to the double bond produced (ethanedithiol). The thus achieved functionalization (thiol group) was utilized for the coupling to biotinyl-iodoacetamidyl-3,6-dioxaoctanediamine, similar to the ICAT method. By means of the biotin, the previously O-phosphorylated peptides can now be subjected to selective affinity purification. The mass-spectrometric analysis after the separation of the peptides allows a relative quantification by means of the peak intensities (Dm=4 Da).

G. Cagney, A. Emili, Nature Biotech, 20(2): 163-70 (2002), describe a method for the labeling of C-terminal lysines in peptides. This method is based on the tryptic digestion of complex protein mixtures, followed by labeling and separation by means of capillary electrophoresis. For detection and quantification, an electrospray tandem mass spectrometer is employed.

A. J. Forbes et al., Proteomics, 1(8): 927-33 (2001), the advantages of Lys-C cleavage over the use of uncleaved proteins for analysis in FT mass spectrometry are explained.

C. S. Spahr et al., Electrophoresis, 21(9): 1635-50 (2000), describe the selective affinity purification of cysteine-containing peptides in a model mixture of proteins and in a model system. The protein sample is biotinylated on existing cysteines by means of commercially available reagents, followed by tryptic digestion. The biotinylated peptides are separated chromatographically by means of a streptavidin column. The thus separated peptides are subsequently released again by the addition of DTT. After alkylation, both the unbound and the bound peptides go to further mass-spectrometric analysis. The latter is performed by LC-MS/MS.

T. J. Griffin et al., Anal. Chem. March 1; 73(5): 978-86 (2001), describe the use of isotope-labeled affinity tags in combination with MALDI-QqTOF mass spectrometry, for the quantitative analysis of complex protein mixtures. The protein mixtures are initially labeled, enzymatically digested and separated by means of multidimensional chromatography in which the elution is effected directly onto the MALDI target. The mass-spectrometric analysis consists in the recording of a mass spectrum of the peptides for determining the intensity relation, followed by fragmentation for sequencing.

DE-A-4344425 describes a method for collecting the aminoterminal peptide fragment of a special protein/polypeptide, in which, after acetylation of α- and ε-amino groups, chemical and/or enzymatic cleavage of the starting protein, the peptide fragments are bound to solid supports through free α-amino groups, and only the unbound peptides are subsequently analyzed.

U.S. 2002/0037532 relates to a method for the analysis of proteins, comprising the cleavage of the proteins by chemical reagents or by enzymes, the coupling of all or part of the fragments to an insoluble support material, washing of the support material, decoupling of the whole fragments bound to the support material, followed by the separation and analysis of the decoupled fragments.

Therefore, it is the object of the present invention to provide a method by which complex mixtures of biopolymers from one or more samples are fragmented, selected by directed coupling to and decoupling from solid support materials, labeled in a sample-specific way, and separated and analyzed after pooling the samples (e.g., quantification and identification/sequencing by mass spectrometry). This invention is supposed to result in low losses of the total information content of the starting polymer mixtures in the analysis, i.e., identification and quantification of their components, as compared to the above stated methods. This is achieved by a directed selection of the biopolymers (biopolymer fragments) by coupling and decoupling reactions as well as fragmentation, which causes a significant reduction of the components to be analyzed without significantly reducing the information content of the starting samples. By sample-specific fluorescence labeling of the biopolymers (biopolymer fragments), a preselection of the molecules to be analyzed by mass spectrometry already during the separating steps is additionally possible, whereby a directed reduction of the components to be analyzed is achieved.

Surprisingly, it has now been found that a significant reduction of the components to be analyzed with a low loss of the total information content of the starting biopolymer mixtures can be achieved especially in the differential analysis of protein and peptide mixtures within the scope of proteome and peptidome analysis by the directed covalent coupling and decoupling of biopolymers or their (chemical or enzymatic) cleavage products to solid support materials (embodiment A). The differential analysis of two or more biopolymer mixtures is achieved by sample-specific labels which exhibit small differences (fluorescence dyes with similar structures, markers with mass differences, for example, by isotope labeling), but which can be detected by measurement. After the separation of the combined biopolymer mixtures (chromatography, electrophoresis etc.) into their components or mixtures of components, the analysis (quantification, identification, characterization) of individual components is effected predominantly by mass spectrometry. By the use of (fluorescence) spectrometers, a directed preselection of fractions in which there are, for example, differences in the intensity, i.e., amounts, of components of the overall mixture is additionally possible due to suitable labels, such as fluorescence markers, after the separation of the mixture. This leads to a reduction of the expenditure in time and cost in mass-spectrometric analysis, since only selected fractions are examined differentially.

As an approach to a solution, there may be mentioned, above all, the directed analysis exclusively of lysine-free peptides (peptide fragments) with a free amino terminus (embodiment B). In this method, all proteins/peptides are first decomposed into fragments by chemical or enzymatic reactions. These fragments are bound to solid support materials through their N-terminal amino groups and, if any, through amino groups from lysines by means of isothiocyanate derivatives. This is followed by the decoupling of biopolymers (fragments) which are bound exclusively through their N-terminal amino group, since the formation of anilinothiazolidinone is possible only in this case. Subsequently, the biopolymers of the thus released mixture of biopolymers are labeled, for example, with fluorescence markers. All sample mixtures treated in parallel and labeled differently (for example, different mass labeling, different fluorescence labeling) are then combined for further differential analysis. This is followed by a separation of the mixtures and analysis by means of spectroscopic and/or mass-spectrometric methods in order to quantify as well as characterize and identify the sample components (inter alia, obtaining sequence information from MS/MS). An advantage of this method is the fact that a large reduction of the fragments to be analyzed with respect to the whole starting cleavage mixture is achieved without significantly reducing the information content with respect to the starting polymer mixtures (proteomes).

In embodiment C, a variant has been invented which allows for a selective analysis of all N termini of proteins and peptides from mixtures. Thus, all the lysines and N-terminal amino groups of the proteins/peptides are first blocked, for example, with citraconic anhydride. This is followed by a cleavage of the proteins/peptides into fragments by chemical or enzymatic reactions. In the next step, by analogy with embodiment B, all polymers which contain amino groups are bound to solid supports. Only the original N termini of the proteins/peptides are not bound and can be eluted and collected for further labeling, separation/analysis. Bound peptides can be selectively decoupled sequentially in addition by performing cleavage of the peptides at methionines by CNBr treatment, or decoupling is effected by analogy with embodiment B. As described above, the further analytical steps (separation and spectroscopic/spectrometric analysis for quantification and characterization/identification) follow after the various peptides of the different starting samples have been labeled in a sample-specific way and combined.

An advantage of this embodiment is the fact that only the N-terminal peptide is analyzed per protein and thus a maximum reduction of the complexity to one peptide per protein is achieved. This cannot be done even approximately with any of the conventional methods.

### SUMMARY OF THE INVENTION

Therefore, the present invention relates to a detection method with the use of chemical and/or biochemical reagents, separation methods and spectrometric as well as mass-spectrometric methods which employ these reagents for analyzing compounds and complex mixtures of compounds. In particular, chemicals and/or biological compounds (e.g., enzymes) are used which react with and fragment biopolymers. In addition, compounds are employed which enable the selective coupling to and decoupling from support materials of these biopolymers.

The polymers or parts of polymers (briefly referred to as “proteins/peptides” or “compounds” in the following) are covalently bound to an insoluble support by using these reagents, and a specific fraction of all bound molecules is released again from the support by the use of specific reagents (detachment of the bond). The released (and other unbound) molecules may further be coupled to molecules which ensure a detection which is not based on the properties of the released molecules, such as fluorescent or radioactive compounds (labeling). The released molecules (e.g., peptides) are characterized in detail, especially with the use of mass-spectrometric methods or other methods which allow to reveal something about the molecular or atomic composition of the molecules. The method described can be employed for the qualitative and quantitative analysis of biopolymers in complex mixtures for quantifying the identity (composition) and quantity of biopolymers in at least two different solutions. The labeling of the unbound compounds can be recurred to for their detection and quantification. The (at least two) complex mixtures of unbound compounds may be mixed, and the different labels allow for assignment of the compound examined to one of the mixed solutions. The different labels further allow for a reduction of the compounds to be analyzed to those which were present in different amounts in different solutions.

In particular, the present invention relates to:
(1) a method for analyzing complex mixtures of biopolymers from one or more samples, comprising the following steps:
   (a) cleaving the biopolymers with one or more chemical reagents and/or one or more enzymes into fragments;
   (b) coupling all or part of the biopolymer fragments and/or biopolymers obtained in step (a) by covalent binding to a linker which is already bound to an anchor group on an insoluble support material;
   (c) washing with a suitable solvent to separate the biopolymer fragments and/or biopolymers not coupled in step (b) from the biopolymer fragments and/or biopolymers coupled to the solid support material in step (b);
   (d) decoupling the biopolymer fragments and/or biopolymers coupled to the insoluble support material by cleaving the bond between the linker and the biopolymer fragment and/or biopolymer or a bond within the coupled biopolymer fragment and/or biopolymer;
   (e) covalently binding a label to particular or all biopolymer fragments not coupled in step (b) or decoupled in step (d) by means of labeling reagents, the labeling allowing for detection of the biopolymer fragment due to that or those chemical or physical property or properties which the biopolymer or one or more of its fragments do not have without the labeling or which they have to a measurably different extent; and
   (f) separating the biopolymer fragments labeled in step (e) due to their characteristic physico-chemical properties with detection of the label; and
(2) a kit for analyzing complex mixtures of biopolymers from one or more samples, comprising at least one of the following components:
   (a) one or more chemical reagents or enzymes for cleaving the biopolymers into fragments as defined in (1);
   (b) one or more reagents for performing the coupling as described in (1);
   (c) one or more insoluble support materials according to (1);
   (d) one or more linkers according to (1);
   (e) one or more solvents for washing according to (1);
   (f) one or more reagents for performing the decoupling as defined in (1);
   (g) one or more reagents for covalently binding a label as defined in (1); and
   (h) one or more devices for separation as defined in (1).

The preferably analyzed biopolymers are those which have peptide bonds, i.e., peptide or protein structures or domains.

### DESCRIPTION OF THE FIGURES

FIG. 1: Schematic representation of the method of “selective peptide exclusion chromatography” for proteome analysis.

FIG. 2: FIG. 2 (FIGS. 2-1 to 2-3 are enlarged segments) shows the MALDI MS spectrum of a sample of four proteins (BSA, lactoglobulin, cytochrome c and myoglobin) which have been subjected to tryptic digestion.

FIG. 3: FIG. 3 (FIGS. 3-1 to 3-3 are enlarged segments) shows a mass spectrum which shows the peptide masses after the decoupling step.

FIG. 4: represents a comparative summary of the number of peptides detected by MALDI MS analyses after tryptic digestion of a protein mixture.

### DETAILED DESCRIPTION OF THE INVENTION

The method of the present invention is intended to combine the advantages of different proteome-analytic methods and additionally allow for a (relative) quantification of the components. Further, the complexity of the mixture of analytes is to be reduced by an intelligent selection of a combination of coupling reagents without reducing the total information content. These objects are achieved as follows:

The complex mixture of biopolymers is fragmented by means of enzymatic or chemical reaction or reactions. Of the resulting fragments, a specific portion is bound to an insoluble support material by covalent binding by means of a suitable chemical reaction. This allows for a separation of the bound fragments. Especially, all fragments which possess binding groups (>99.9%) are bound to an insoluble support material. Specific fragments are released by selective decoupling. The bound fragments are detached from the solid phase and subjected to further analysis. Labeling is effected (fluorescence, isotope-labeling, chiral or magnetic labeling). The mixtures of labeled fragments of at least two samples can be combined. A chromatographic separation results in separation into individual fragments (independently of the labeling). These fragments can now be analyzed spectroscopically (only quantitative analysis) and/or by mass spectrometry (quantitative analysis and fragmentation analysis). The properties of the labeling are as follows: The labeling is covalent, and the labeling groups or labeling compounds of the (at least) two samples possibly show identical physico-chemical properties so as to affect chromatographic separation to the same extent.

“Biopolymers” within the meaning of the present invention may be proteins and peptides as well as nucleic acid polymers, lipids, sugars and PNAs (peptide nucleic acids). The proteins may include all conceivable natural modifications whose properties can also be utilized in the further process.

“Complex mixture of biopolymers” within the meaning of the present invention refers to mixtures of biopolymers with more than three components. The “cleavage” or “fragmentation” of the biopolymers of the present invention comprises the treatment of the biopolymers with “cleavage reagents” to obtain “fragments” of the biopolymers.

The “cleavage reagents” according to the present invention enable the cleaving of individual or several amino acids and/or nucleotides and/or their derivatives (e.g., natural modifications due to glycosylation) singly or in chains (as a peptide or nucleic acid) from the original biopolymer selectively, i.e., at particular positions of the respective sequence, or non-selectively. In the case of proteins, the cleavage reagents include, in particular, endoproteases, by the catalytic action of which proteins are degraded into peptides. Endoproteases suitable according to the present invention include trypsin, submaxillaris protease, chymotrypsin, Staphylococcus aureus V8 protease, Asp-N protease, pepsin, Lys-C, Glu-C, Arg-C proteinase, Asp-N endopeptidase, BNPS skatoles, caspases, chymotrypsin, clostripain, factor Xa, glutamyl endopeptidase, granzyme B, proline endopeptidase, proteinase K, Staphylococcus peptidase I, thermolysin, thrombin, carboxypeptidases and a combination thereof. As reagents for chemical cleavage, there may be mentioned, for example, CNBr, formic acid, iodosobenzoic acid, NTCB (2-nitro-5-thiocyano-benzoic acid), hydroxylamine, acid hydrolysis etc., which are key components in the production of fragments from biopolymers without using endoproteinases. For the cleavage of nucleic acids and PNAs, there may be employed, for example, the following endonucleases:

Aat II, Acc65 I, Acc I, Aci I, Acl I, Afe I, Afl II, Afl III, Age I, Ahd I, Alu I, Alw I, AlwN I, Apa I, ApaL I, Apo I, Asc I, Ase I, AsiS I, Ava I, Ava II, Avr II, Bae I, BamH I, Ban I, Ban II, Bbs I, Bbv I, BbvC I, BceA I, Bcg I, BciV I, Bcl I, Bfa I, BfrB I, BfuA I, Bgl I, Bgl II, Blp I, Bme1580 I, BmgB I, Bmr I, Bpm I, BsaA I, BsaB I, BsaH I, Bsa I, BsaJ I, BsaW I, BsaX I, BseR I, Bsg I, BsiE I, BsiHKA I, BsiW I, Bsl I, BsmA I, BsmB I, BsmF I, Bsm I, BsoB I, Bsp1286 I, BspCN I, BspD I, BspE I, BspH I, BspM I, BsrB I, BsrD I, BsrF I, BsrG I, Bsr I, BssH II, BssK I, BssS I, BstAP I, BstB I, BstE II, BstF5 I, BstN I, BstU I, BstX I, BstY I, BstZ17 I, Bsu36 I, Btg I, Bts I, Cac8 I, CIa I, Dde I, Dpn I, Dpn II, Dra I, Dra III, Drd I, Eae I, Eag I, Ear I, Eci I, EcoN I, EcoO109 I, EcoR I, EcoR V, Fau I, Fnu4H I, Fok I, Fse I, Fsp I, Hae II, Hae III, Hga I, Hha I, Hinc II, Hind III, Hinf I, HinP1 I, Hpa I, Hpa II, Hpy188 I, Hpyl88 III, Hpy99 I, HpyCH4III, HpyCH4IV, HpyCH4V, Hph I, Kas I, Kpn I, Mbo I, Mbo II, Mfe I, Mlu I, Mly I, Mnl I, Msc I, Mse I, Msl I, MspA1 I, Msp I, Mwo I, Nae I, Nar I, Nci I, Nco I, Nde I, NgoM IV, Nhe I, Nla III, Nla IV, Not I, Nru I, Nsi I, Nsp I, Pac I, PaeR7 I, Pci I, PflF I, PflM I, Ple I, Pme I, Pml I, PpuM I, PshA I, Psi I, PspG I, PspOM I, Pst I, Pvu I, Pvu II, Rsa I, Rsr II, Sac I, Sac II, Sal I, Sap I, Sau3A I, Sau96 I, Sbf I, Sca I, ScrF I, SexA I, SfaN I, Sfc I, Sfi I, Sfo I, SgrA I, Sma I, Sml I, SnaB I, Spe I, Sph I, Ssp I, Stu I, Sty I, Swa I, Taq I, Tfi I, Tli I, Tse I, Tsp45 I, Tsp509 I, TspR I, Tth111 I, Xba I, Xcm I, Xho I, Xma I, Xmn I, N.BstNB I, N.Alw I, I-Ceu I, I-Sce I, PI-Psp I, PI-Sce I, McrBC endonuclease, McrBC etc.

In the case of sugars, amylases, maltases and lactases may be employed.

“Fragments” within the meaning of the present invention means any compounds formed from the cleavage of a biopolymer by means of enzymes or chemical reagents and included, in particular, in one or more classes of compounds selected from the groups of amino acids, peptides, nucleotides, nucleic acids, lipids, sugars and their derivatives. In particular, “derivatives” means fragments which bear a label or result from a blocking reaction. “Blocking” within the meaning of the present invention means that particular monomers are derivatized by chemical reagents or enzymes at position n within the sequence of the biopolymer in such a manner that the linkage between this blocked monomer and the monomer immediately upstream (at position n−1) and/or downstream (at position n+1) in the sequence will not be cleaved during fragmentation. The term “blocking” may further mean that a derivatization of one or both terminal monomers (termini) of the biopolymer is effected by chemical reagents or enzymes prior to coupling so that the blocked termini will not be coupled to the solid support material during the coupling. Suitable chemical reagents for blocking include, in particular, acid halides, acid anhydrides, aldehydes, isocyanate derivatives, isothiocyanate derivatives, succinimide derivatives, imidazolyl carbamate derivatives, Traut's reagent derivatives, sulfonic chloride derivatives, oxirane derivatives, imidates, hydrazides, sulfosuccinimidyl derivatives, diimide derivatives, maleimide derivatives, 7-sulfobenzofurazan derivatives, especially acetyl chloride, and citraconic anhydride.

The “coupling” within the meaning of the present invention is a chemical reaction in which the biopolymer or one or more of its fragments is covalently bound to a suitable “insoluble support material”, said binding to the insoluble support material being preferably effected through a linker which is bound to the insoluble support material through an anchor group. The covalent coupling preferably produces stable compounds under reductive conditions, for example, amides, esters, carbon-heteroatom bonds etc. (but no S—S bonds).

The “insoluble support material” or “solid phase” within the meaning of the present invention includes a suitable material which is insoluble in the solvent employed (e.g., activated glass surfaces, magnetic beads and polymer materials with functional groups) as known in the prior art for solid-phase syntheses of peptides and nucleic acids, especially a resin, such as polystyrene. The insoluble support material is preferably provided with a suitable anchor group, which is a functional group reactive under the conditions of the method according to the invention, so that a suitable linker can be bound to the anchor group.

Suitable as the “anchor group” within the meaning of the present invention are all forms of functional groups which result in an activated support material, especially —NH₂, —SH, hydrazides, tosyl, tresyl, imidazolylcarbamate, 5-thiol-2-nitrobenzoic acid groups, or also CNBr-activated support material.

The “linker” within the meaning of the present invention is a compound with two identical or different functional groups reactive under the conditions of the method according to the invention, of which one functional group, X¹, enables binding to the anchor group and the other functional group, X², enables binding to the biopolymer or one or more of its fragments. As functional groups for the linker according to the invention, there may be used two identical or different functional groups X¹ and X². These may be selected from —NH₂, —CN, —OH, —COOH, —COCl, —CON₃, —CHO, —NN, —SH, —SCH₃, —NNH, —CHCH₂, —NCS, —NCO, —CNO, —CNS, —SO₂Hal, —OPO₃²⁻, oxirane and vinylsulfone. Also suitable are mixed anhydrides and active esters wherein X¹ and/or X² are selected from —C(O)OR, R being selected from R¹C(O)—, ortho-nitrophenyl, —C(NR¹)(NHR¹), N-oxysuccinimide and 1-oxybenzotriazole, R¹ being selected from lower alkyl, cycloalkyl, aryl, alkenyl and alkynyl.

In particular, the linker may have the formula X¹-(A)ₙ-X², wherein A represents an aryl, heteroaryl, alkyl, CH₂ structure, silyl, ether or thioether, n is a natural number of from 1 to 20, and X¹ and X² are as defined above. Particularly preferred as a linker is 1,4-diisothiocyanatobenzene (para-diisothiocyanatobenzene, pDITC).

The coupled biopolymer within the meaning of the present invention may have the following formula: 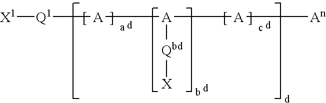
wherein X=a solid phase
A=an amino acid or amino acid derivative or monomer of another biopolymer (sugar; lipid, nucleoside, nucleotide etc.)
Q=a linker
a≧0
b≧0
c≧0
d≧0
n≧Σ_{d}(a^{d}+b^{d}+c^{d}).

Q is preferably a mono- or bifunctional linker which is covalently coupled to the amino acid (or the respective monomer component of the biopolymer) and/or covalently coupled to the solid phase. The linkage of Q to A may be effected through one of the following chemical bonds: N—N, C—C, N—C, C—S, N—S, S—S, C—O, N—O, S—O, O—O, P—O, P—N, P—C, P—S.

The linkage of Q to X may also be effected through one of the chemical bonds mentioned below. For the formation of the chemical bond, the properties of the respective amino acids are utilized. For the formation of the linkage, the linker Q disposes of at least one reactive group of the form: —NH₂, —CN, —OH, —COOH, —CHO, —NN, —SH, —S—CH₃, —N═NH, —C═CH₂, —N═C═S, —N═C═O, —C═N═O, —C═N═S, —SO₂Cl, —COCl, oxirane, vinylsulfone.

The above mentioned structure may further be linked with markers ((fluorescent) dyes, isotope labels and the like). The linker may include or be an oligonucleotide, PNA or peptide.

The “washing” comprises one or more cycles of adding and removing a liquid or the continuous adding and removing of a liquid to remove components of the starting solution. If desired, these liquids may be subjected to further analysis.

“Decoupling” within the meaning of the present invention means that the biopolymer fragments and/or biopolymers coupled to the insoluble support material are detached from the support material by cleaving the bond between A) the support material and the linker, B) within the linker, C) between the linker and the biopolymer (fragment), and/or D) within the biopolymer (fragment). “Cleavage” means the cleavage of one or more of bonds 1, 2, 3 and/or 4 (Scheme 1, A) and the cleavage of a bond within the linker (B) and/or within the biopolymer or biopolymer fragment (C). This is further illustrated in the following Scheme 1.

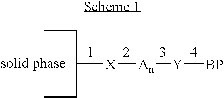

A: Numbering scheme of the bonds for the coupling of the biopolymer or biopolymer fragment through a linker within the meaning of the invention.

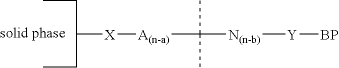

B: Cleavage of a bond within the linker. A is any of the molecular groups mentioned above, n is a whole number, n, a, b<20, and a+b=m.

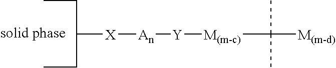

C: Cleavage of a bond within the biopolymer or biopolymer fragment. M are any of the monomers mentioned above of the biopolymer or biopolymer fragment; m, c, d are natural numbers≧0, and c+d=m.

The “labeling” is effected by the chemical reaction of a biopolymer or one or more of its fragments with the labeling reagent, wherein the biopolymer or one or more of its fragments is coupled with the labeling reagent. The labeling provides the biopolymer or one or more of its fragments with a chemical or physical property which the biopolymer or one or more of its fragments did not have previously, or which they had to a lesser extent, i.e., to a measurably different extent as compared to the labeled state.

The “labeling reagent” may be exemplified by (fluorescent) dyes, but there may also be mentioned, in particular, isotope-labeled, chiral or magnetic compounds, and combinations of the mentioned labeling properties within the same labeling reagent are also possible according to the invention.

“Separation” means the separation of the individual labeled fragments by means of electrophoretic and/or chromatographic methods, preferably by means of liquid chromatography (LC), more preferably by means of high performance liquid chromatography (HPLC) on reverse phases, such as C18 reverse phase, or ion-exchange chromatography.

“Characterization” and “identification” includes the structural elucidation of the labeled fragments with spectroscopic methods, for which mass spectroscopy, NMR, UV, Vis and IR spectroscopies are suitable.

“Device for separation” means a system for liquid chromatography, capillary electrophoresis, zone electrophoresis, gel electrophoresis, free-flow electrophoresis, extraction.

“Device for characterization” means one or more devices for the detection of the introduced label (e.g., fluorescence detector, absorption spectrometer, multiphoton detector) and/or the structure of a biopolymer or biopolymer fragment (mass spectrometer, NMR).

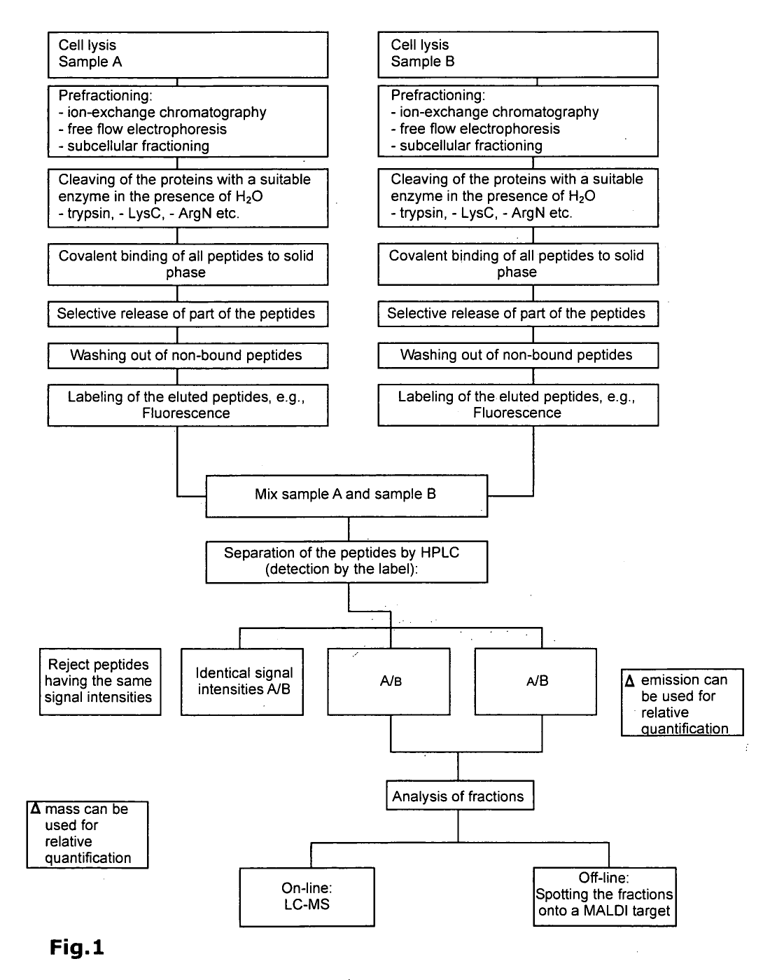

Embodiment A of the method of the present invention employs “selective peptide-exclusion chromatography” for proteome analysis and comprises the following steps (see also the schematic representation in FIG. 1):

A.1. Fractioning of the Complex Mixtures of Biopolymers:

This step is performed optionally, depending on the complexity of the analyte or the separation capacity of the subsequent steps. Suitable methods include chromatographic methods as well as physico-chemical separation methods.
   A.2. Cleavage of the Biopolymers:
The biopolymers of the complex mixture are fragmented. This is effected, for example, by the use of enzymes. Another possibility is the chemical fragmentation of the biopolymers to be analyzed, e.g., with CNBr or partial acidic hydrolysis.
The cleavage of the biopolymer or mixture of biopolymers may be effected, on the one hand, with the aid of the catalytic action of other purified biopolymers or by the activation of biopolymers which are present in the solution. Purified biopolymers, if used, may be employed both in a soluble form and in a form coupled to an insoluble material.
Another possibility is the use of chemical reagents for the cleavage of the biopolymers contained in the mixture. This may be exemplified by the use of CNBr, by which proteins are selectively cleaved into fragments according to their primary sequence.
   A.3. Retardation of the Biopolymer Fragments:

Fragments resulting from step 2 are covalently coupled to an insoluble support material. The coupling reaction may be specific or non-specific in nature, i.e., all or only part of the fragments resulting from step 2 may be coupled to the material. Homo- or heterobifunctional reagents may be employed as coupling reagents. By the coupling to the insoluble support material, it is possible to handle the biopolymer fragments in insoluble form.
The coupling is achieved by means of chemical reagents and suitable reaction conditions. The kind of coupling may be matched to some special property of the biopolymer, or coupling may be effected selectively through a particular component of the biopolymer. The coupling may be performed with a mono- or heterobifunctional reagent. It does not matter whether the coupling of the linker is done first to an insoluble support material and then to a biopolymer, or in the reverse order. Phenylenediisothiocyanate may be exemplified as a coupling reagent.
In addition, the coupling reagent may directly contain a group which unequivocally labels the biopolymer, due to either its specific physicochemical properties or its mass or other characteristics. The following methods may be employed in step A.3:
a) Examples of the reaction of amines (—NH₂)
reaction with anhydrides: 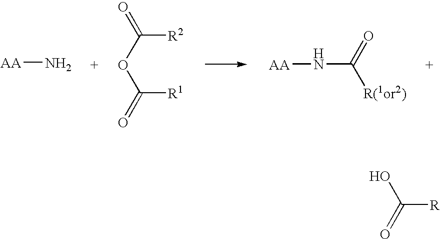
succinimide esters: 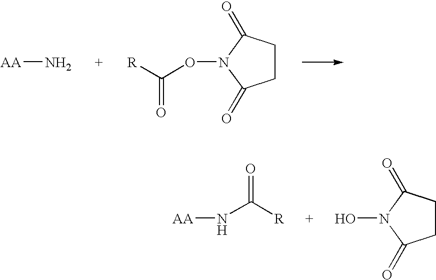
aminidation: 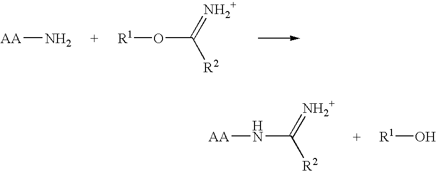
(reductive) alkylation: 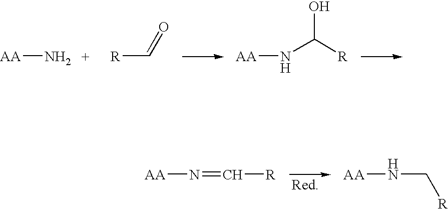
halogen carbamates, e.g.: 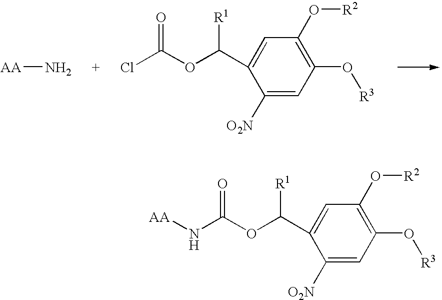
b) Examples of the reaction of thiols
disulfides, such as 5,5′-dithiobis(2-nitrobenzoic acid) (DTNB, Ellman reagent): 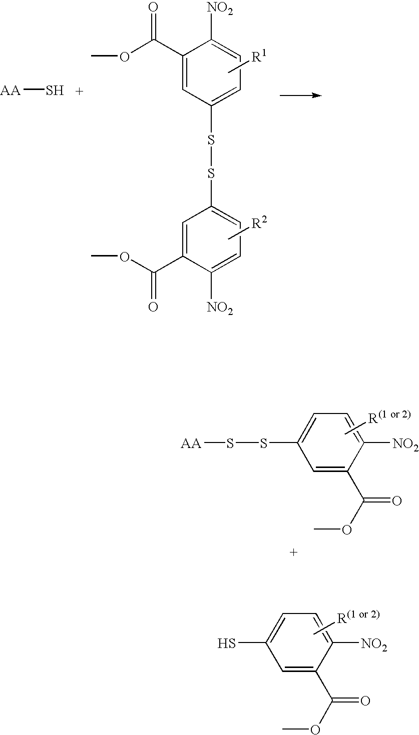
reaction with halogen-mercury compounds: 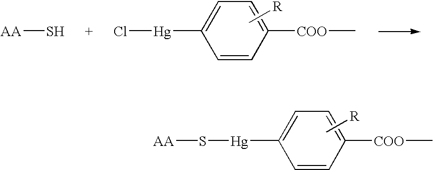
reaction with 2-nitro(hydroxy)benzyl bromide: 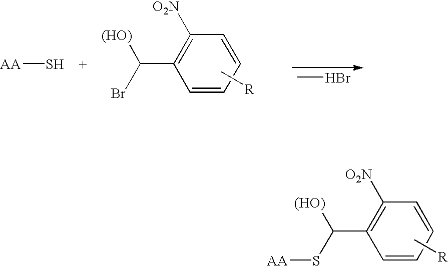
c) Examples of the reaction of glutamate residues or aspartate residues (carboxy groups)
reaction with carbodiimides: 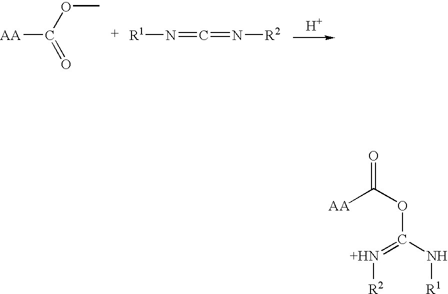
d) Examples of the reaction of arginine residues
reaction with glyoxal (derivatives) 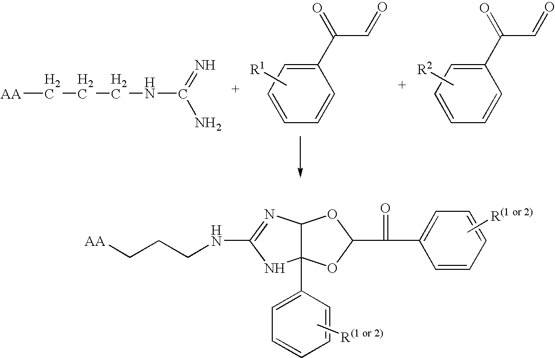
e) Examples of the reaction of tyrosine residues
Koshland's reagent (derivatives): 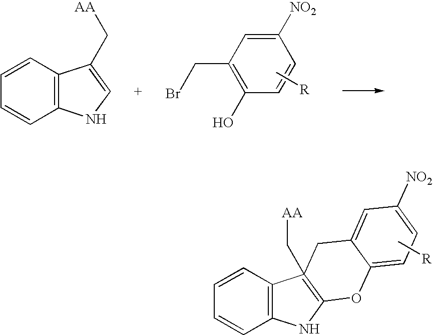
reaction with sulfenyl halides 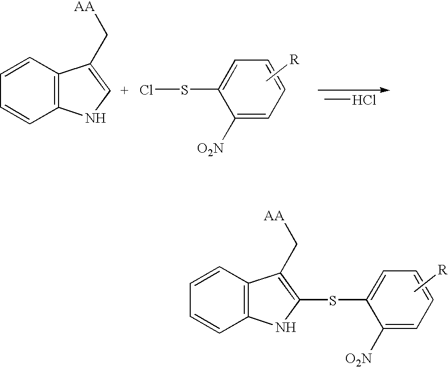
f) Examples of the reaction of methionine with iodoacetamide derivatives (reversible) 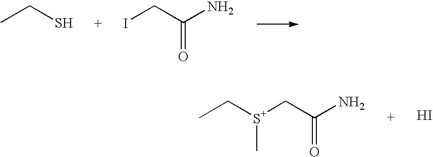
g) Examples of the reaction of histidine residues
reaction with diethyl pyrocarbonate (derivatives)

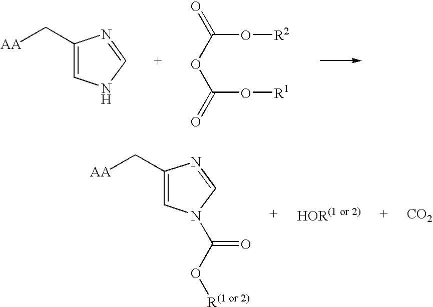

Coupling reagents within the meaning of the invention also include silicon compounds of the following type as long as they are employed for the functionalization of glass surfaces (matrix): 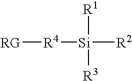
a. This primarily means compounds in which R¹=R²=R³=—O—(CH₂)ₙH, with n≧1, and R⁴=—(CH₂)ₘH, with m≧1. Further, RG is a reactive group, which means a functional group as described above by means of which either a linker or a biopolymer or biopolymer fragment can be bound to the silane compound.
b. Each R is any residue desired which either bears further functional groups and/or is coupled to a matrix within the meaning of the invention.
c. If several residues are present in the examples (R², R³ etc.), any of them may be the same or different. Further, each of the residues meets the conditions mentioned under a).
   A.4. Decoupling of the Biopolymers:
The biopolymers coupled to the insoluble support material can be decoupled selectively or non-specifically. Both the nature of the coupling and the method employed in the production of the fragments are important. By selecting the fragmentation, coupling and decoupling method, the complexity in biopolymers of the mixture is reduced. As shown in Scheme 1, the decoupling of the biopolymers can be effected between the support material and linker, within the linker, between the linker and the biopolymer (fragment), or within the biopolymer (fragment). The latter is possible by an endoprotease cleavage or CNBr cleavage.
   A.5. Labeling of the Fragments:
The fragments of the biopolymers are provided with labels which allow for subsequent assignment to the starting sample.
   A.6. Analysis of the Labeled Biopolymer Fragments:
The labeled biopolymer fragments are separated from each other by characteristic physico-chemical properties. Previously, labeled biopolymer fragments from different samples may be mixed.
   A.7. Analysis of the Separated Biopolymer Fragments:
The separation of the biopolymer fragments (step 6) may also be subsumed herein. Further analytical methods primarily include mass spectrometry by means of which an exact characterization of the biopolymer fragments is effected. The latter allows for assignment to the original biopolymer.

According to need, steps A.2, A.3 and A.5 can be performed in a different order. Thus, for example, it is conceivable to perform the labeling before the fragmenting by which a functional group for covalently binding the biopolymer is introduced. The covalent coupling in turn may be performed both before and after the fragmentation.

Biomolecules not bound in step A.3 may also be subjected to further labeling and analysis from step A.5.

In addition to the labeling of biopolymers in step A.5, it is also possible to label the biopolymers during the cleavage (A.2) by using heavy water and/or [¹⁸O]water. This variant can be employed for the differential analysis of two starting samples, optionally instead of labeling by A.5, by cleaving one sample in the presence of H₂O and the other in the presence of heavy water or [¹⁸O]water or heavy [¹⁸O]water. Analysis is then performed in steps A.6 and A.7 by mass spectrometry.

In the preferred embodiment B, the present invention relates to a method for isolating peptides from complex protein, peptide or other mixtures which comprises the following steps B.1 to B.6 with the cleavage-coupling-decoupling sequence of reactions of the biopolymer mixtures. This embodiment is suitable for the analysis of non-lysine-containing peptides from fragmented proteins. It has the advantage that N-terminal fragments of proteins blocked in the first step (B.4) can be isolated and subjected to further differential analytics. In step B.5, all lysine-free peptides are decoupled and subjected to further differential analytics. This has the advantage that only a reduced number of peptides per protein must be subjected to differential analytics while the protein coverage is significantly higher than that in the above described method ICAT.

B.1. Cleavage of the Proteins in the Protein Mixture

B.2. Activation with pDITC of the Solid Phase Provided with an —NH₂ Functionality: 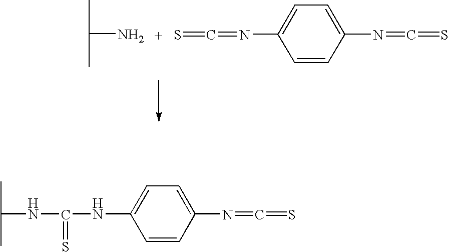
B.3. Coupling of the Peptides to the Insoluble Support Material: 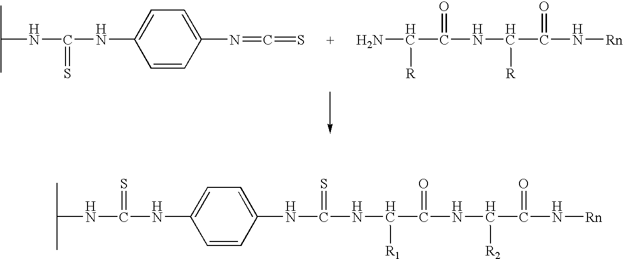
B.4. Washing Steps (Collecting the Non-Coupled Peptides)
B.5. Decoupling: 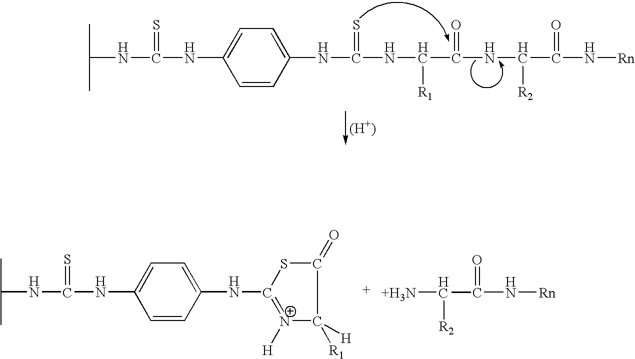
B.6. Labeling:
In the further course of the process, the decoupled and eluted peptides are labeled. This is preferably done through the free N termini, which are necessarily present, by using a labeling reagent.

In step B.3, all peptides having a free N-terminus are coupled to the matrix. Thus, the non-coupled peptides obtained in step B.4 correspond to the blocked N termini of those proteins which bear an arginine upstream of a lysine in the sequence. These are also isolated and analyzed since they represent a major part of the proteins initially employed.

In step B.4, the non-coupled biopolymer fragments are additionally isolated and subjected to further analysis.

In step B.5, only those peptides can be decoupled which are coupled exclusively through a free N terminus, i.e., under these conditions, the decoupling in step B.5 is effected only if the formation of an ATZ (anilinothiazolidinone) is possible, so that only those peptides are released which are coupled to the insoluble support material exclusively through a free N terminus. Peptides which are coupled to the insoluble support material through amino acid side chains having NH₂ functionality (Lys), cannot be released by means of this sequence of reactions. Thus, a suited combination of cleavage reagent and coupling reagent enables a selective reduction of the peptide mixture to a sufficiently small amount of peptides.

As a labeling reagent for step B.6, a fluorescence marker is exemplified in the following. The physico-chemical properties of fluorescence markers should not be very different, whereas their emission maxima should be as far apart as possible.

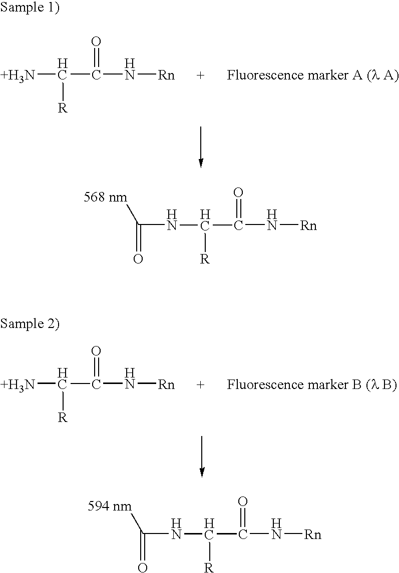

The thus labeled samples are now combined and separated by LC, so that ideally identical peaks per fluorescence peak are co-eluted.

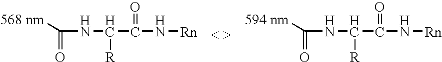

Now, due to the fluorescence labeling, the relative ratio of the peptides can be determined, and only those fractions which have different fluorescence intensities are subjected to analysis. Thus, the number of fractions to be analyzed is significantly reduced.

The fact that blocked N termini with arginine residues at the C terminus are not coupled can be utilized for further specification of the analysis. For example, the mixture of proteins is blocked synthetically, which can be done, for example, by acetylation or by reaction with citraconic acid or citraconic anhydride (as shown in FIG. 1), followed by tryptic digestion. (i.e., with trypsin) or other enzymatic or chemical cleavage. Thus, peptides which only result from the cleavage dispose of a free N terminus through which they can be coupled to a matrix. The blocked N-terminal peptides are not coupled to the solid matrix and can be analyzed. In this method, the protein coverage should be 100% theoretically.

In the further more preferred embodiment C of the method, the isolation of the N termini of all proteins of a sample is effected according to steps C.1 to C.4 as shown in the following. Characteristic of this embodiment C is the analysis of (synthetically) blocked N termini of proteins and blocking, e.g., with citraconic anhydride. This process variant has the advantage that only the N termini of proteins are analyzed (i.e., only one peptide per protein is analyzed with a protein coverage of about 100%, while in the above described ICAT method, only all the cysteine-containing peptides are analyzed). It is to be noted that about 16% of the proteins which are listed in the NCBI protein data base do not contain any cysteine and thus cannot be detected by cysteine-selective methods. The remaining 84% of the proteins in the NCBI protein data base contain at least one cysteine, and many of them contain several cysteines. This means that the ICAT method must analyze a clearly higher number of peptides, which results in a clearly higher complexity of the method.
C.1 Blocking of N Termini and Lysine Residues by Reaction with Aldehydes, Isocyanate, Isothiocyanates, N-hydroxysuccinimide Ester Group, Sulfonic Halides, Activated Esters or Acid Anhydrides, Especially Citraconic Anhydride, for Example, According to: 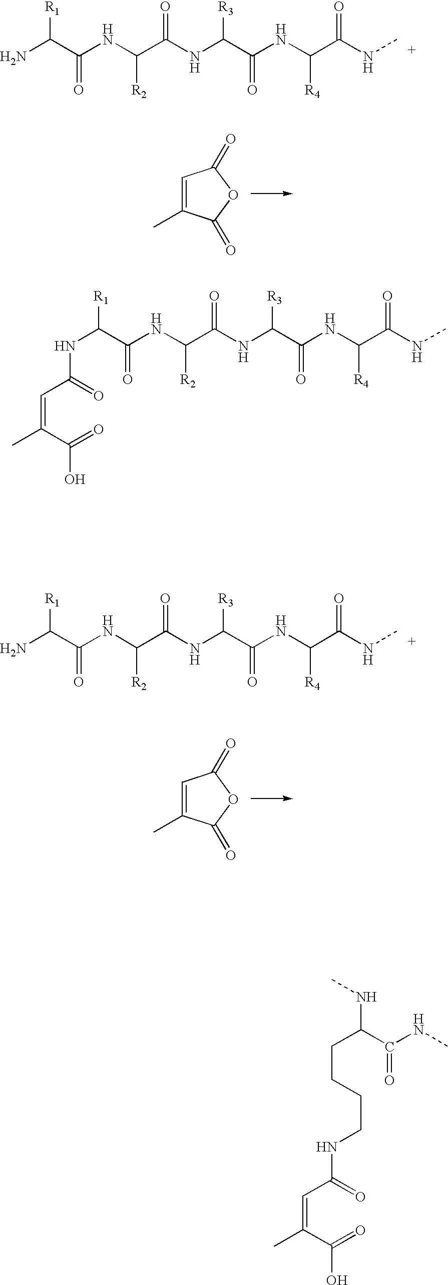
C.2 Cleavage with Trypsin (Only at the C-Terminal Side of Arg)
C.3 Coupling to Insoluble Support Material/Matrix (—R—N═C═S)

Thus, all the N termini are first blocked. After the cleavage, all internal and C-terminal fragments again have a free N terminus which can be coupled to the matrix. The only peptides which are not coupled to the matrix are the N termini. These are fractioned by HPLC and analyzed in a mass spectrometer. The prior labeling and mixing of different samples is optional.

However, a further reduction of the number of peptides is also possible, wherein the reactivity of methionine can be utilized, in particular. Thus, it is conceivable to perform the cleavage of the biopolymers with other enzymes or chemically (combined cleavage). The number of peptides obtained should be altered drastically thereby. However, it is also conceivable that the peptides coupled to the matrix by the method described are chemically reacted with CNBr, so that peptides formed by the cleavage of methionine are eluted from the matrix in this case. For example, a quasi two-step extraction of the matrix-coupled peptides can be effected thereby, since the non-lysine-containing peptides can still be eluted (by degradation by analogy with Edman chemistry) after CNBr cleavage. In this way, the process can be split into differently sensitive subprocesses which can be applied to different organisms or problems.

Another embodiment of the method according to the invention includes the utilization of the lactone intermediate occurring during the CNBr reaction. This intermediate may also be utilized for. the coupling of the peptides to a solid matrix. Thus, the homoserine lactone is formed in the following reaction steps:
Reaction 1—Reaction of a Methionine-Containing Biopolymer with CNBr: 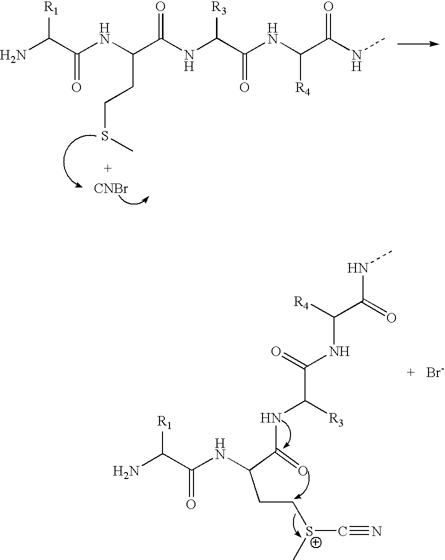
Reaction 2—Formation of the Iminolactone: 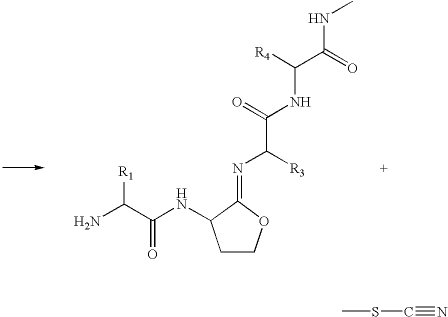
Reaction 3—Cleavage of the C-terminal Residue and Formation of a Homoserine Lactone: 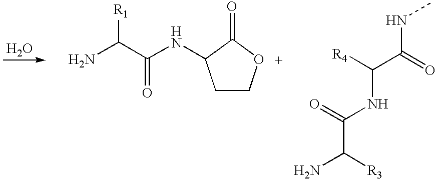
Reaction 4—Equilibrium Reaction between Homoserine Lactone and C-Terminal Homoserine:

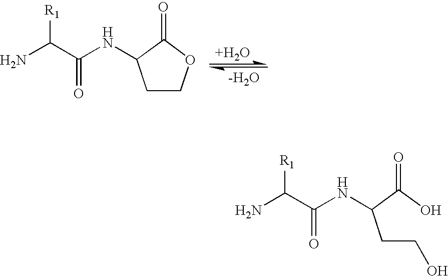

The (homoserine) lactone ring is an active component and can be reacted with amines; quantitative reaction is ensured because the lactone is withdrawn from the equilibrium by the reaction.

The method according to the invention has the following differences from the ICAT method:

A) The elementary difference of the method according to the invention from the ICAT method is the fact that the biopolymers are NOT provided with a detectable affinity tag. The biopolymers are selected by inherent properties.

B) Order of the process steps:
ICAT: labeling-mixing-selection-analysis
Method according to the invention: selection-labeling-mixing-analysis

C) The method according to the invention is based on a negative selection whereas ICAT is based on a positive selection (labeled peptides are separated off by affinity chromatography and then analyzed by LC-MS).

D) The method according to the invention has a higher variability because the selective decoupling can be effected chemically or enzymatically. The coupling may also be effected selectively.

E) The selection of the method according to the invention is NOT effected by affinity (biotin/avidin), but by the formation of a covalent bond (not equilibrium-dependent).

F) The method according to the invention employs chemical methods which have become established and validated in peptide chemistry for many years.

Due to these differences, the method according to the invention includes many possibilities for specification in the following process steps:

A) Pretreatment of the sample for increasing the selectivity (chemical blocking of all N termini, see preferred embodiment C, chemical modification of cleavage sites).

B) Generation of the fragments by means of enzymatic or chemical cleavage.

C) Covalent coupling of the fragments through different functionalities (see Examples).

D) Selective decoupling of the fragments (see Scheme 1, matrix-X-linker-Y-biopolymer).

The present invention is further illustrated by the following Examples, which are not to be construed as limiting the scope of the invention, however.

### EXAMPLES

Materials and Methods

Reagents: NH₂-functionalized support material (e.g., aminopropyl glass, APG), toluene, pDITC, DMF, THF, TFA, MeOH, protective gas, acetic acid, HCI, triethyl-amine (TEA)

Buffer B: 0.2 M Na2HPO4, pH 9.0, 1% SDS

Coupling of the Linker to the Matrix:

take up about 200 mg of DITC in 5 ml of dry DMF under protective gas;

add 2 g of aminopropyl glass beads;

incubate for 2 h;

suck off the solvent; and

wash the glass beads with 100 ml of benzene;

subsequently wash the glass beads with 150 ml of anhydrous MeOH;

dry under vacuum; and

store at 4° C.

Coupling of the Biopolymer to the Linker/Washing:

dry the biopolymer;

resuspend the biopolymer in buffer B;

add a suitable amount of APG/DITC support material and TEA;

heat at 55° C. for 45 minutes;

wash out non-bound components with buffer B, 20% TFA and water (optionally subjected to analysis with separation and MS).

Decoupling (By Analogy with Current Edman Chemistry) and Labeling:

dry the solid phase (APG-DITC-BP);

decoupling with TFA;

dry the liquid phase (contains free BP or BP fragments) in SpeedVac;

reacting with F-SCN (F=fluorescent residue) in THF, 30 min, 52° C., where each sample obtains a different fluorescent residue (e.g., different emission maxima, different masses, different isotope labeling);

drying;

taking up in aqueous solution.

Mix the samples to be compared and subject them to the subsequent separation and analysis.

Separation: Subsequently, the combined mixtures of the labeled biopolymers or biopolymer fragments are separated by means of chromatographic or eledrophoretic methods. Detection is effected with suitable detectors (e.g., fluorescence detector and mass detector).

### Example

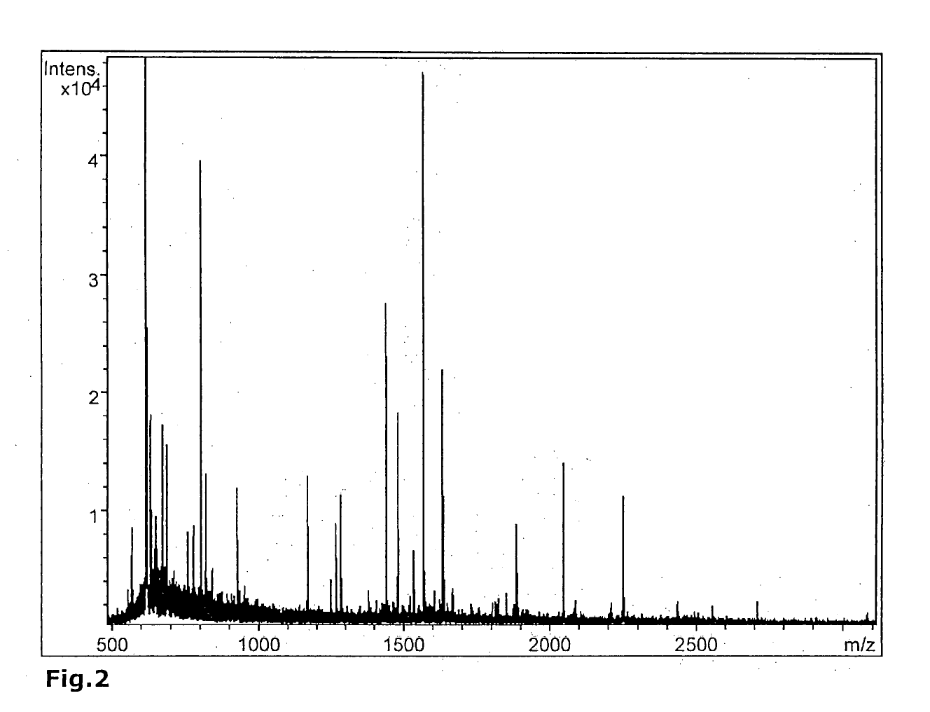

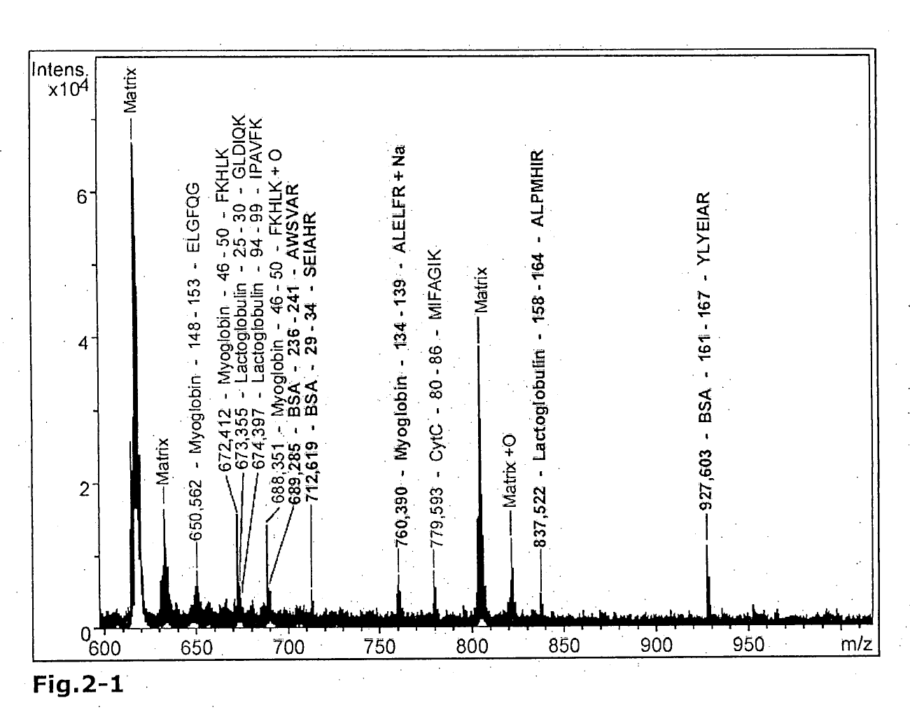

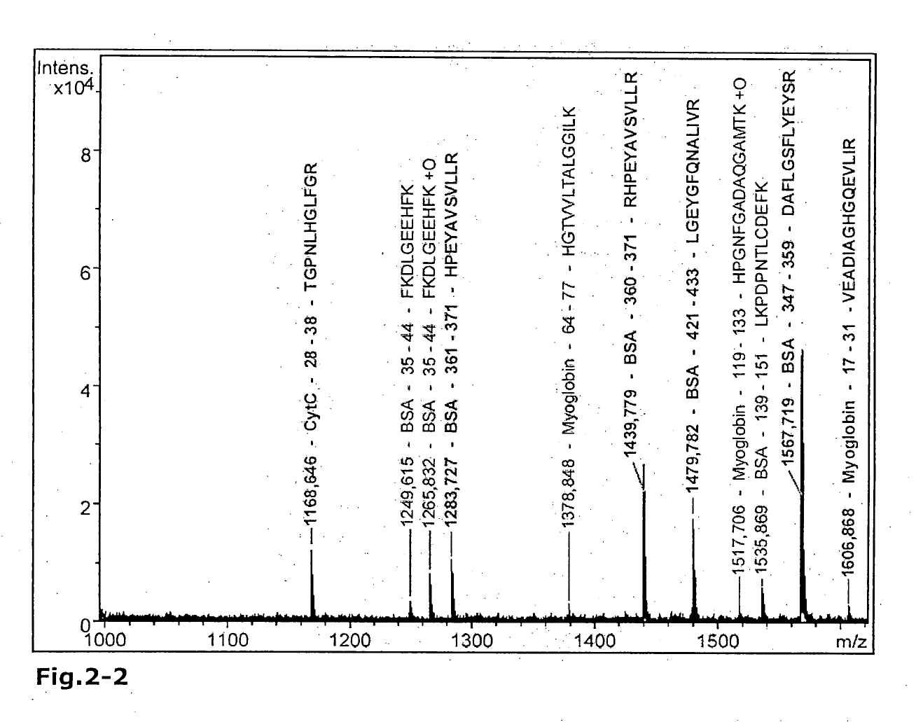

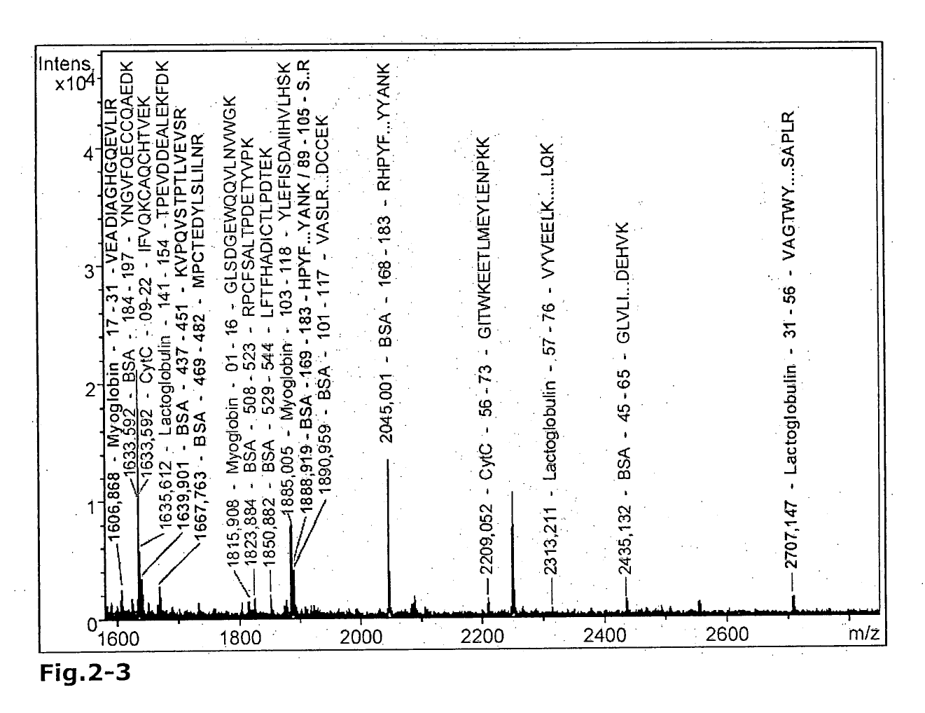

FIG. 2 (FIGS. 2-1 to 2-3 are enlarged segments) shows the MALDI MS spectrum of a sample of four proteins (BSA, lactoglobulin, cytochrome c and myoglobin) which have been subjected to tryptic digestion. The peptides were subsequently coupled to DITC-APG beads. This involves a non-reversible covalent binding of the N-terminal α-amino groups of the peptides and of the ε-amino groups of lysines within the peptides to the beads. After intensive washing steps for the removal of non-bound peptides (e.g., of blocked N-terminal peptides from proteins which do not contain any lysine), the selective decoupling of bound peptides which do not have a lysine residue is effected. Thus, the N-terminal amino acid is cleaved off as an ATZ amino acid derivative, and the lysine-free peptides are thus decoupled from the beads. The covalent binding between DITC and the coupled amino groups of the peptides is not reversible, but can be cleaved off as an AZT amino acid derivative by ring formation of the N-terminal amino acid of the PTC peptide derivative in the case of the a amino group, so that the peptide residue is no longer bound to the beads through the N terminal.

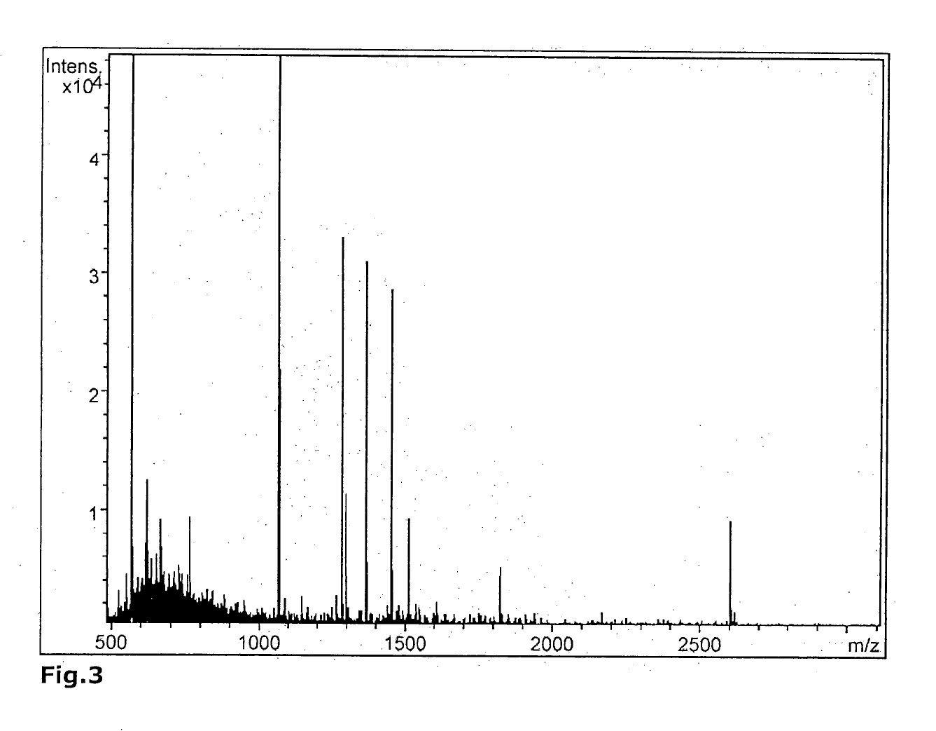

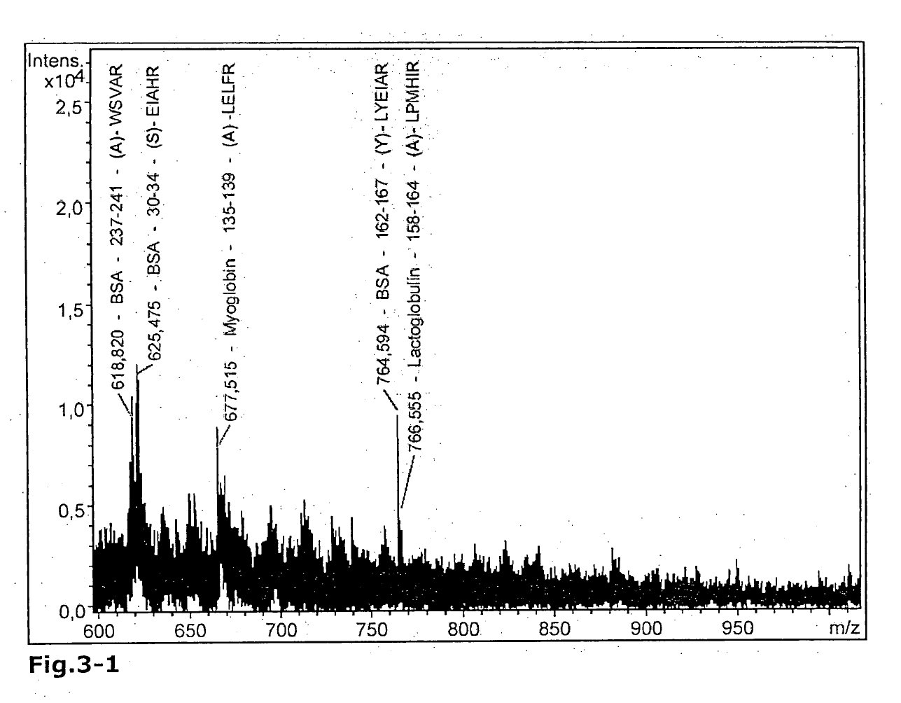

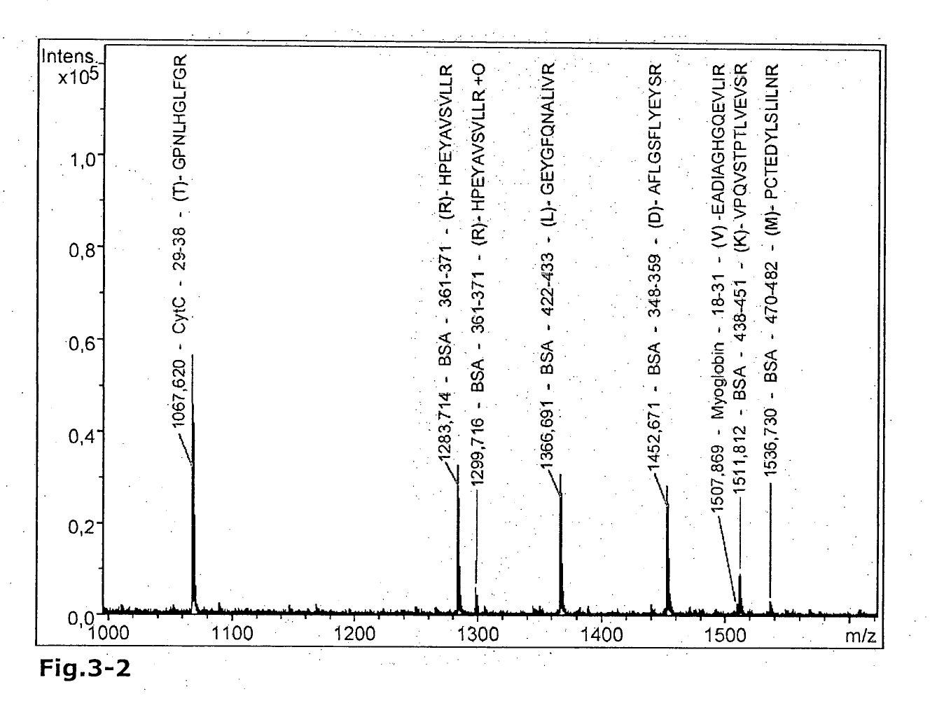

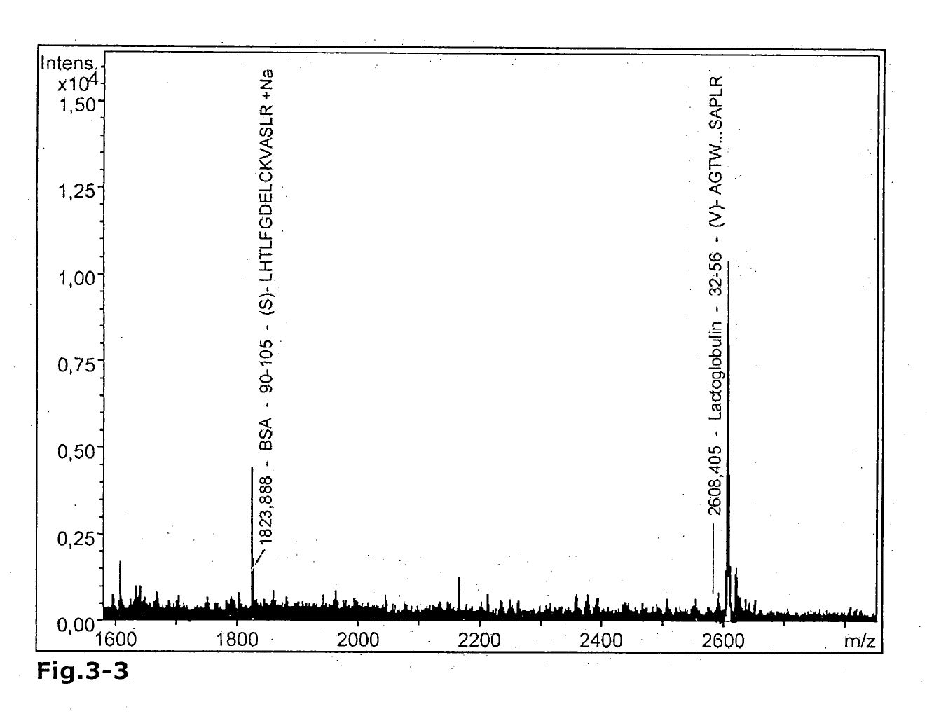

FIG. 3 (FIGS. 3-1 to 3-3 are enlarged segments) shows a mass spectrum which shows the peptide masses after the decoupling step. From the inscription (mass, amino acid sequence), it can be seen that only lysine-free peptides were detected in which the N-terminal amino acid has been cleaved off during the decoupling step. The result is a significant reduction of the complexity in peptide masses. Identification of the proteins is ensured by MS/MS analyses of the peptides.

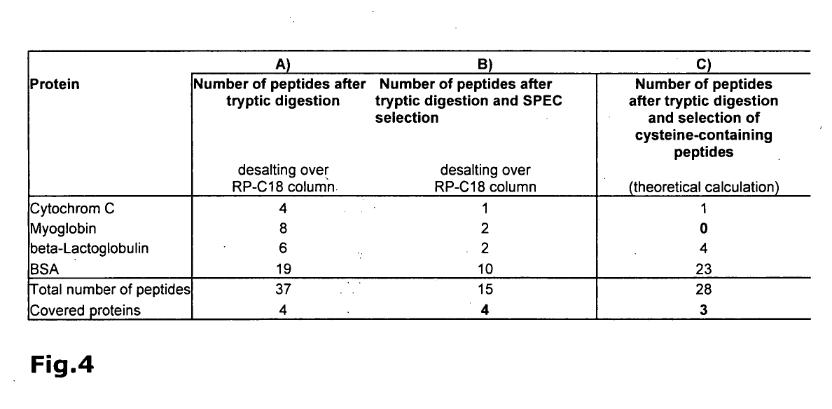

FIG. 4 represents a comparative summary of the number of peptides which could be detected by MALDI MS analyses after tryptic digestion of a protein mixture (cytochrome c, myoglobin, β-lactoglobulin and BSA) and

A): desalting over RP C18 column;

B): peptide selection over coupling/decoupling on DITC support material (SPEC)+desalting over RP C18 column; and

C): theoretical selection of cysteine-containing peptides.

It is obvious that peptides from all four proteins could be detected by the SPEC selection (B), while only three proteins can be identified theoretically via the selection of cysteine-containing peptides (C) since one protein does not contain any cysteine. This holds for about 15-20% of all proteins, depending on the respective organism. At the same time, a reduction of the number of measured peptides by about 60% as compared to the starting digestion (A) was achieved in the SPEC method. In contrast, in the selection of cysteine-containing peptides, clearly more peptides, namely 28 peptides, would be detected.

1. A method for analyzing complex mixtures of biopolymers with peptide bonds from one or more samples, comprising the following steps:
(a) cleaving the biopolymers with one or more chemical cleaving reagents and/or one or more enzymes into fragments;
(b) coupling all or part of the biopolymer fragments and/or biopolymers containing amino acids as obtained in step (a) by covalent binding to a linker which is already bound to an anchor group on an insoluble support material;
(c) washing with a suitable solvent to separate the biopolymer fragments and/or biopolymers not coupled in step (b) from the biopolymer fragments and/or biopolymers coupled to the insoluble support material in step (b);
(d) selectively decoupling the biopolymer fragments and/or biopolymers coupled to the insoluble support material by cleaving the bond between the linker and the biopolymer fragment and/or biopolymer or a bond within the coupled biopolymer fragment and/or biopolymer;
(e) covalently binding a label to particular or all biopolymer fragments not coupled in step (b) or decoupled in step (d) by means of labeling reagents, the labeling allowing for detection of the biopolymer fragment due to that or those chemical or physical property or properties which the biopolymer or one or more of its fragments do not have without the labeling or which they have to a measurably different extent; and
(f) separating the biopolymer fragments labeled in step (e) due to their characteristic physico-chemical properties with detection of the label.

2. The method according to claim 1, wherein:
(i) the biopolymers are selected from one or more members of the group of peptides/proteins, peptide-nucleic acids (PNAs), lipoproteins/-peptides, glycopeptides/-proteins and their derivatives, and/or which are bound to the support material;
fragments are selected from one or more members of the group of amino acids, peptides, PNAs, lipopeptides, glycopeptides and their derivatives; and/or
(ii) said covalent binding of the biopolymer to the linker is a covalent bond which is stable under reductive reaction conditions; and/or
(iii) the kind and number of the chemical cleaving reagents and enzymes in step (a) are selected to produce at least two different fragments which can be bound to the support material in step (b).

3. The method according to claim 1, wherein:
(i) prior to the cleavage (step (a)), a step for fractioning the complex biopolymer mixtures after the cell lysis by chemical and/or physico-chemical separation methods is inserted, preferably by one or more methods selected from subcellular fractioning, precipitation, free electrophoresis and chromatographic methods, especially ion-exchange chromatography, size-exclusion chromatography (gel filtration) and affinity chromatography; and/or
(ii) prior to the cleavage (step (a)), the blocking of particular monomers at position n within the sequence of the biopolymer is effected, so that the linkage between this blocked monomer and the monomer immediately upstream (at position n−1) and/or downstream (at position n+1) in the sequence will not be cleaved in step (a); and/or
(iii) prior to the coupling (step (b)), the blocking of one or both terminal monomers (termini) of the biopolymer is effected, so that the blocked termini are not coupled to the solid support material in step (b).

4. The method according to claim 1, wherein:
(i) the coupling in step (b) is effected first to the free linker, and then the adducts of biopolymer fragments and linker and/or adducts of biopolymer and linker are bound to the anchor group of the insoluble support material through a functional group of the linker; and/or
(ii) the linker is a compound with two identical or different functional groups reactive under the conditions of the method according to the invention, of which one functional group enables binding to the anchor group and the other functional group enables binding to the biopolymer or one or more of its fragments; and/or
(iii) the linker has two identical or different functional groups X¹ and X² selected from —NH₂, —CN, —OH, —COOH, —COCl, —CON₃, —CHO, —NN, —SH, —SCH₃, —NNH, —CHCH₂, —NCS, —NCO, —CNO, —CNS, —SO₂Hal, —OPO₃²⁻, oxirane and vinylsulfone, preferably having the formula X¹-(A)ₙ-X², wherein A represents an aryl, heteroaryl, alkyl group, CH₂ structure, silyl, ether or thioether structure, and n is a natural number of from 1 to 20, A preferably additionally including an amino acid, peptide, nucleoside, nucleic acid or PNA residue, or A is an amino acid, peptide, nucleoside or nucleic acid residue.

5. The method according to claim 1, wherein:
(i) the washing solution obtained in step (c) is collected, and the biopolymer fragments and/or biopolymers contained in the washing solution are subjected to independent analysis and/or again subjected to step (a) and subsequently to the further process steps; and/or
(ii) for each mixture of biopolymer fragments which originate from a particular sample of biopolymers, a particular label is selected in step (e), so that the thus labeled biopolymer fragments can be assigned to the respective sample, wherein the labels for different samples have negligibly different or identical influences on the separation behavior of the labeled biopolymer fragments, wherein preferably a fluorescent dye, an isotope-labeled, chiral and/or magnetic compound is bound as a label to the biopolymer fragment in step (e); and/or
(iii) in step (f), the labeled biopolymer fragments are additionally separated in such a way that only those fractions are subjected to the characterization in step (g) which have different intensities of the signal detected due to the labeling, thus achieving a reduction of the number of fractions to be employed in step (g); and/or labeled biopolymer fragments from different starting samples are mixed prior to the separation in step (f).

6. The method according to claim 1, wherein steps (a) (cleavage), (b) (coupling) and (e) (labeling) are performed in a different order, preferably:
(i) step (b) is performed prior to step (a), so that the coupling of the biopolymers to the solid support material is effected prior to the cleavage; and/or
(ii) step (e) is performed prior to step (a), so that biopolymers which have already been labeled are fragmented, and more preferably the coupling in step (b) is effected through the label.

7. The method according to claim 1, wherein characterization of the labeled biopolymer fragments separated in step (e) is effected by spectrometric methods, especially by mass spectrometry, said characterization being suitable for determining the sequence of a peptide, a protein, a nucleic acid or a PNA.

8. The method according to claim 1, wherein proteins and/or peptides as biopolymers and peptides and/or amino acids and/or their derivatives as biopolymer fragments are subjected to the method, wherein in step (a):
one or more enzymes, especially those selected from trypsin, submaxillaris protease, chymotrypsin, Staphylococcus aureus V8 protease, Asp-N protease, pepsin, Lys-C, Glu-C, Arg-C proteinase, Asp-N endopeptidase, BNPS skatoles, caspases, chymotrypsin, clostripain, factor Xa, glutamyl endopeptidase, granzyme B, proline endopeptidase, proteinase K, Staphylococcus peptidase I, thermolysin, thrombin, carboxypeptidases and a combination thereof, are used; and/or
one or more chemical reagents selected from acid, especially hydrochloric acid, TFA and amino acid, phenyl isothiocyanate and cyanogen bromide are used.

9. The method according to claim 1, wherein:
(i) methionine and/or peptides containing methionine, after reaction with cyanogen bromide, is coupled by reaction of the homoserine lactone formed with an amine group of the linker or with iodoacetamide derivatives or with an amine group on the insoluble support material; and/or
(ii) primary amine groups are coupled to the insoluble support material by reaction with anhydrides, isothiocyanates, succinimide esters or halogen carbamates, by aminidation or by (reductive) alkylation; and/or
(iii) thiols are bound to the insoluble support material by reaction with disulfides, halogen-mercury compounds or 2-nitro(hydroxy)benzyl bromide; and/or
(iv) glutamate or aspartate residues are bound to the insoluble support material by reaction with carbodiimides; and/or
(v) arginine residues are bound to the insoluble support material by reaction with glyoxal or glyoxal derivatives; and/or
(vi) tyrosine residues are bound to the insoluble support material by reaction with Koshland's reagent or sulfenyl halides; and/or
(vii) histidine residues are bound to the insoluble support material by reaction with diethyl pyrocarbonate or a derivative thereof.

10. The method according to claim 1, wherein: prior to the cleavage (step (a)) the blocking of particular monomers at position n within the sequence of the biopolymer is effected, so that the linkage between this blocked monomer and the monomer immediately upstream (at position n−1) and/or downstream (at position n+1) in the sequence will not be cleaved in step (a) and wherein
(i) the blocking is effected by reacting the biopolymer with a reagent selected from acid halides, acid anhydrides, aldehydes, isocyanate derivatives, isothiocyanate derivatives, succinimide derivatives, imidazolyl carbamate derivatives, Traut's reagent derivatives, sulfonic chloride derivatives, oxirane derivatives, imidates, hydrazides, sulfosuccinimidyl derivatives, diimide derivatives, maleimide derivatives, 7-sulfobenzofurazan derivatives, especially acetyl chloride, and citraconic anhydride; and/or
(ii) the linker has the formula X¹-(A)ₙ-X², wherein A represents an aryl, heteroaryl, alkyl group, CH₂ structure, silyl, ether or thioether structure, n is a natural number of from 1 to 20, and X¹ and X² are identical or different functional groups of formula —C(O)OR, R being selected from RC(O)—, ortho-nitrophenyl, —C(NR¹)(NHR¹), N-oxysuccinimide and 1-oxybenzotriazole, R¹ being selected from lower alkyl, cycloalkyl, aryl, alkenyl and alkynyl; and/or
(iii) 1,4-diisothiocyanatobenzene is used as a linker in step (b); and/or
(iv) a resin, especially polystyrene, is employed as an insoluble support material; and/or
(v) the decoupling is effected by reducing the pH value, so that only N-terminally bound peptides are decoupled, and an ATZ (anilinothiazolidinone) which is bound to the insoluble support material is present after decoupling; and/or
(vi) the label in step (e) has fluorescence properties suitable for detection in step (f);
and/or
(vii) the separation in step (f) is effected by chromatography, preferably by liquid chromatography (LC), more preferably by high-performance liquid chromatography (HPLC) on reverse phases.

11. The method according to claim 1 which is suitable for analyzing non-lysine-containing peptides from fragmented proteins and which comprises the steps of:
(a) isolating blocked N-terminal fragments from proteins; and
(b) decoupling of all lysine-free peptides, wherein the fractions obtained in (a) and (b) are subjected to further differential analytics.

12. The method according to claim 1 which is suitable for analyzing (synthetically) blocked N-termini of proteins, preferably those blocked with citraconic anhydride, in which only the N termini of proteins are analyzed.

13. A kit for analyzing complex mixtures of biopolymers from one or more samples according to the method of claim 1, comprising the following components:
(a) one or more chemical reagents or enzymes for cleaving the biopolymers into fragments;
(b) one or more reagents for performing the coupling; and
(c) one or more reagents for performing the selective decoupling.

14. The kit according to claim 13, further comprising one or more of the following components:
(d) one or more insoluble support materials;
(e) one or more linkers as;
(f) one or more solvents for washing;
(g) one or more reagents for covalently binding a label; and
(h) one or more containers with one or more reagents for blocking wherein the blocking reagent is selected from the group consisting of acid halides, acid anhydrides, aldehydes, isocyanate derivatives, isothiocyanate derivatives, succinimide derivatives, imidazolyl carbamate derivatives, Traut's reagent derivatives, sulfonic chloride derivatives, oxirane derivatives, imidates, hydrazides, sulfosuccinimidyl derivatives, diimide derivatives, maleimide derivatives, 7-sulfobenzofurazan derivatives, especially acetyl chloride, and citraconic anhydride.


SEQUENCE LISTING (52 sequences)

SEQ ID NO: 1 - protein, 6 aa, Artificial sequence
  Description of artificial sequence; note = synthetic construct
ELGFQG

SEQ ID NO: 2 - protein, 5 aa, Artificial sequence
  Description of artificial sequence; note = synthetic construct
FKHLK

SEQ ID NO: 3 - protein, 6 aa, Artificial sequence
  Description of artificial sequence; note = synthetic construct
GLDIQK

SEQ ID NO: 4 - protein, 6 aa, Artificial sequence
  Description of artificial sequence; note = synthetic construct
IPAVFK

SEQ ID NO: 5 - protein, 5 aa, Artificial sequence
  Description of artificial sequence; note = synthetic construct
FKHLK

SEQ ID NO: 6 - protein, 6 aa, Artificial sequence
  Description of artificial sequence; note = s ynthetic constructt
AWSVAR

SEQ ID NO: 7 - protein, 6 aa, Artificial sequence
  Description of artificial sequence; note = synthetic construct
SEIAHR

SEQ ID NO: 8 - protein, 6 aa, Artificial sequence
  Description of artificial sequence; note = synthetic construct
ALELFR

SEQ ID NO: 9 - protein, 7 aa, Artificial sequence
  Description of artificial sequence; note = synthetic construct
MIFAGIK

SEQ ID NO: 10 - protein, 7 aa, Artificial sequence
  Description of artificial sequence; note = synthetic construct
ALPMHIR

SEQ ID NO: 11 - protein, 7 aa, Artificial sequence
  Description of artificial sequence note = synthetic construct
YLYEIAR

SEQ ID NO: 12 - protein, 11 aa, Artificial sequence
  Description of artificial sequence; note = synthetic construct
TGPNLHGLFGR

SEQ ID NO: 13 - protein, 10 aa, Artificial sequence
  Description of artificial sequence; note = synthetic construct
FKDLGEEHFK

SEQ ID NO: 14 - protein, 11 aa, Artificial sequence
  Description of artificial sequence; note = synthetic construct
HPEYAVSVLLR

SEQ ID NO: 15 - protein, 14 aa, Artificial sequence
  Description of artificial sequence; note = synthetic construct
HGTVVLTALGGILK

SEQ ID NO: 16 - protein, 12 aa, Artificial sequence
  Description of artificial sequence; note = synthetic construct
RHPEYAVSVLLR

SEQ ID NO: 17 - protein, 13 aa, Artificial sequence
  Description of artificial sequence; note = synthetic construct
LGEYGFQNALIVR

SEQ ID NO: 18 - protein, 15 aa, Artificial sequence
  Description of artificial sequence; note = synthetic construct
HPGNFGADAQGAMTK

SEQ ID NO: 19 - protein, 13 aa, Artificial sequence
  Description of artificial sequence; note = synthetic construct
LKPDPNTLCDEFK

SEQ ID NO: 20 - protein, 13 aa, Artificial sequence
  Description of artificial sequence; note = synthetic construct
DAFLGSFLYEYSR

SEQ ID NO: 21 - protein, 15 aa, Artificial sequence
  Description of artificial sequence; note = synthetic construct
VEADIAGHGQEVLIR

SEQ ID NO: 22 - protein, 14 aa, Artificial sequence
  Description of artificial sequence; note = synthetic construct
YNGVFQECCQAEDK

SEQ ID NO: 23 - protein, 14 aa, Artificial sequence
  Description of artificial sequence; note = synthetic construct
IFVQKCAQCHTVEK

SEQ ID NO: 24 - protein, 14 aa, Artificial sequence
  Description of artificial sequence; note = synthetic construct
TPEVDDEALEKFDK

SEQ ID NO: 25 - protein, 15 aa, Artificial sequence
  Description of artificial sequence; note = synthetic construct
KVPQVSTPTLVEVSR

SEQ ID NO: 26 - protein, 14 aa, Artificial sequence
  Description of artificial sequence; note = synthetic construct
MPCTEDYLSLILNR

SEQ ID NO: 27 - protein, 16 aa, Artificial sequence
  Description of artificial sequence; note = synthetic construct
GLSDGEWQQVLNVWGK

SEQ ID NO: 28 - protein, 16 aa, Artificial sequence
  Description of artificial sequence; note = synthetic construct
RPCFSALTPDETYVPK

SEQ ID NO: 29 - protein, 16 aa, Artificial sequence
  Description of artificial sequence; note = synthetic construct
LFTFHADICTLPDTEK

SEQ ID NO: 30 - protein, 17 aa, Artificial sequence
  Description of artificial sequence; note = synthetic construct
YLEFISDAHIIHVLHSK

SEQ ID NO: 31 - protein, 15 aa, Artificial sequence
  Variant (5)..(11): Xaa can be any amino acid
  Description of artificial sequence; note = synthetic construct
HPYFXXXXXXXYANK

SEQ ID NO: 32 - protein, 17 aa, Artificial sequence
  Variant (2)..(16): Xaa can be any amino acid
  Description of artificial sequence; note = synthetic construct
SXXXXXXXXXXXXXXXR

SEQ ID NO: 33 - protein, 17 aa, Artificial sequence
  Variant (6)..(12): Xaa can be any amino acid
  Description of artificial sequence; note = synthetic construct
VASLRXXXXXXXDCCEK

SEQ ID NO: 34 - protein, 16 aa, Artificial sequence
  Variant (6)..(11): Xaa can be any amino acid
  Description of artificial sequence; note = synthetic construct
RHPYFXXXXXXYYANK

SEQ ID NO: 35 - protein, 18 aa, Artificial sequence
  Description of artificial sequence; note = synthetic construct
GITWKEETLMEYLENPKK

SEQ ID NO: 36 - protein, 20 aa, Artificial sequence
  Variant (8)..(17): Xaa can be any amino acid
  Description of artificial sequence; note = synthetic construct
VYVEELKXXXXXXXXXXLQK

SEQ ID NO: 37 - protein, 21 aa, Artificial sequence
  Variant (6)..(16): Xaa can be any amino acid
  Description of artificial sequence; note = synthetic construct
GLVLIXXXXXXXXXXXDEHVK

SEQ ID NO: 38 - protein, 26 aa, Artificial sequence
  Variant (7)..(21): Xaa can be any amino acid
  Description of artificial sequence; note = synthetic construct
VAGTWYXXXXXXXXXXXXXXXSAPLR

SEQ ID NO: 39 - protein, 5 aa, Artificial sequence
  Description of artificial sequence; note = synthetic construct
WSVAR

SEQ ID NO: 40 - protein, 5 aa, Artificial sequence
  Description of artificial sequence; note = synthetic construct
EIAHR

SEQ ID NO: 41 - protein, 5 aa, Artificial sequence
  Description of artificial sequence; note = synthetic construct
LELFR

SEQ ID NO: 42 - protein, 6 aa, Artificial sequence
  Description of artificial sequence; note = synthetic construct
LYEIAR

SEQ ID NO: 43 - protein, 6 aa, Artificial sequence
  Description of artificial sequence; note = synthetic construct
LPMHIR

SEQ ID NO: 44 - protein, 10 aa, Artificial sequence
  Description of artificial sequence; note = synthetic construct
GPNLHGLFGR

SEQ ID NO: 45 - protein, 11 aa, Artificial sequence
  Description of artificial sequence; note = synthetic construct
HPEYAVSVLLR

SEQ ID NO: 46 - protein, 12 aa, Artificial sequence
  Description of artificial sequence; note = synthetic construct
GEYGFQNALIVR

SEQ ID NO: 47 - protein, 12 aa, Artificial sequence
  Description of artificial sequence; note = synthetic construct
AFLGSFLYEYSR

SEQ ID NO: 48 - protein, 14 aa, Artificial sequence
  Description of artificial sequence; note = synthetic construct
EADIAGHGQEVLIR

SEQ ID NO: 49 - protein, 14 aa, Artificial sequence
  Description of artificial sequence; note = synthetic construct
VPQVSTPTLVEVSR

SEQ ID NO: 50 - protein, 13 aa, Artificial sequence
  Description of artificial sequence; note = synthetic construct
PCTEDYLSLILNR

SEQ ID NO: 51 - protein, 16 aa, Artificial sequence
  Description of artificial sequence; note = synthetic construct
LHTLFGDELCKVASLR

SEQ ID NO: 52 - protein, 25 aa, Artificial sequence
  Variant (5)..(20): Xaa can be any amino acid
  Description of artificial sequence; note = synthetic construct
AGTWXXXXXXXXXXXXXXXXSAPLR